(19) United States
(12) Patent Application Publication
(10) Pub. No.: US 2024/0352053 A1
(43) Pub. Date: Oct. 24, 2024

(54) SALT AND CRYSTAL FORM OF EGFR INHIBITOR, AND COMPOSITION AND USE THEREOF

(71) Applicant: BETTA PHARMACEUTICALS CO., LTD, Hangzhou (CN)

(72) Inventors: Liang CHEN, Hangzhou (CN); Changyong QIU, Beijing (CN); Xiangyong LIU, Beijing (CN); Jian TANG, Hangzhou (CN); Lieming DING, Hangzhou (CN); Jiabing WANG, Beijing (CN)

(73) Assignee: BETTA PHARMACEUTICALS CO., LTD, Hangzhou (CN)

(21) Appl. No.: 18/683,571

(22) PCT Filed: Aug. 19, 2022
(86) PCT No.: PCT/CN2022/113456

§ 371 Date: Feb. 14, 2024

(30) Foreign Application Priority Data
Aug. 19, 2021 (WO) PCT/CN2021/113458

(51) Int. Cl. C07F 9/6558; A61K 31/675; C07C 55/10; C07C 57/15; C07C 59/245; C07C 59/255; C07C 309/04

(52) U.S. Cl. CPC C07F 9/65583; A61K 31/675; C07C 55/10; C07C 57/15; C07C 59/245; C07C 59/255; C07C 309/04; C07B 2200/13

(57) ABSTRACT

The present invention relates to salt and crystal form of an EGFR inhibitor, and a composition and the use thereof. The salt and crystal form of the EGFR inhibitor as represented by formula I of the present invention can be used for treating or preventing epidermal growth factor receptor-mediated diseases or medical conditions (such as L858R activation mutants, exon 19 deletion activation mutants, T790M resistance mutants and C797S resistant mutants) in certain mutant forms.

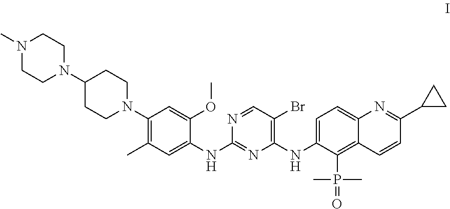 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1]

### FIELD OF THE INVENTION

The invention belongs to the field of medicine, and specifically relates to a salt, crystal form of an EGFR inhibitor and its composition and application. The salts and crystal forms of the EGFR inhibitor of the present invention can be used to treat or prevent diseases or medical conditions mediated by certain mutant forms of epidermal growth factor receptor (for example, L858R activating mutants, Exon19 deletion activating mutants, T790M resistance mutant and C797S-resistant mutant).

### BACKGROUND OF THE INVENTION

Epidermal growth factor receptor (EGFR) is a transmembrane glycoprotein that belongs to the ErbB family of tyrosine kinase receptors. Activation of EGFR leads to autophosphorylation of receptor tyrosine kinases and participates in the cascade of downstream signaling pathways that regulate cell proliferation, differentiation, and survival. EGFR is abnormally activated by various mechanisms, such as receptor overexpression, mutation, ligand-dependent receptor dimerization, and ligand-independent activation, and is associated with the development of various human cancers.

PCT international application PCT/CN2021/075994 describes a class of quinolylphosphine oxide compounds used as EGFR inhibitors. Most of these compounds can effectively inhibit EGFR. Since there are still unmet needs in treatment options for EGFR-mediated diseases, here we further screen the salts and crystal forms of quinolylphosphine oxide compounds as EGFR inhibitors to meet the medical needs of patients.

### SUMMARY OF INVENTION

The object of the present invention is to provide a crystal form of the compound shown in formula I:

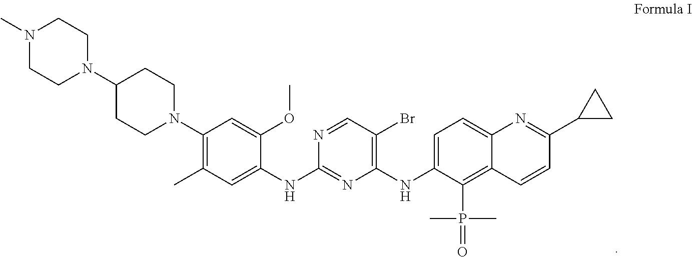 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1]

In some embodiments, the crystal form is selected from one or more of Form α, Form β, Form γ, and Form δ.

In some embodiments, the X-ray powder diffraction pattern of Form α is an X-ray powder diffraction pattern substantially as shown in FIG. 1.

In some embodiments, the crystal form α is substantially pure, and its crystal form purity is ≥85%; further, the crystal form purity is ≥95%; further, the crystal form purity is ≥99%; further, the purity of the crystal form is ≥99.5%.

In some embodiments, the crystalline form β is characterized by having an X-ray powder diffraction pattern comprising characteristic peak in term of angle 2θ at 4.7±0.2°, 10.3±0.2°, 11.2±0.2°, 11.6±0.2°, 13.1±0.2°, 13.3±0.2°, 14.5±0.2°, 17.5±0.2°, 18.6±0.2°, 18.9±0.2°, 19.7±0.2°, 20.3±0.2°, 21.4±0.2°, 21.8±0.2°; further, all The X-ray powder diffraction pattern of the crystal form β is basically shown in FIG. 2.

In some embodiments, the crystal form β is substantially pure, and its crystal form purity is ≥85%; further, the crystal form purity is ≥95%; further, the crystal form purity is ≥99%; further, the purity of the crystal form is ≥99.5%.

In some embodiments, the crystalline form γ is characterized by having an X-ray powder diffraction pattern comprising characteristic peak in term of angle 2θ at 4.8±0.2°, 7.6±0.2°, 9.8±0.2°, 10.0±0.2°, 11.6±0.2°, 19.8±0.2°, 4.8±0.2°, 7.6±0.2°, 9.8±0.2°, 10.0±0.2°, 11.6±0.2°, 14.3±0.2°, 14.8±0.2°, 15.5±0.2°, 19.1±0.2°, 19.5±0.2°, 19.8±0.2°, 20.0±0.2°, 22.2±0.2°, 23.1±0.2°, 23.9±0.2°; further, the X-ray powder diffraction pattern of the crystal form γ is basically shown in FIG. 3.

In some embodiments, the crystal form γ is substantially pure, and its crystal form purity is ≥85%; further, the crystal form purity is ≥95%; further, the crystal form purity is ≥99%; further, the purity of the crystal form is ≥99.5%.

In some embodiments, the crystalline form δ is characterized by having an X-ray powder diffraction pattern comprising characteristic peak in term of angle 2θ at 5.9±0.2°, 8.2±0.2°, 9.6±0.2°, 10.7±0.2°, 11.2±0.2°, 15.7±0.2°, 21.8±0.2; further, the X-ray powder diffraction pattern of the crystal form δ is basically shown in FIG. 4.

In some embodiments, the crystalline form δ is substantially pure, and its crystalline purity is ≥85%; further, the crystalline purity is ≥95%; further, the crystalline purity is ≥99%; further, the purity of the crystal form is ≥99.5%.

A composition, the composition comprising a therapeutically effective amount of the crystal form of the compound represented by formula I; further, the crystalline form is selected from one or more of said crystalline form α, crystalline form β, crystalline form γ and crystalline form δ.

In some embodiments, the composition further includes pharmaceutically acceptable excipients.

A method for inhibiting various forms of EGFR mutations, including one or more of the L858R, Δ19del, T790M and C797S mutations, the method comprises administering a crystal form of the compound shown in formula I to patients or the composition containing a therapeutically effective amount of the crystal form of a compound shown in formula I; further, the crystal form is selected from one or more of the above-mentioned crystal form α, crystal form β, crystal form γ and crystal form δ.

A method for treating EGFR driven cancer, comprising administering a crystal form of the compound shown in formula I or a composition containing a crystal form of the compound shown in formula I in a therapeutically effective amount to patients in need; further preferably, the crystal form is selected from one or more of the above-mentioned crystal form α, crystal form β, crystal form γ and crystal form δ.

In some embodiments, EGFR driven cancers are characterized by the presence of one or more mutations selected from: (i) C797S, (ii) L858R and C797S, (iii) C797S and T790M, (iv) L858R, T790M, and C797S, (v) Δ19del, T790M and C797S, (vi) Δ19del and C797S, (vii) L858R and T790M, or (viii) Δ19del and T790M.

In some embodiments, the EGFR-driven cancer is colon cancer, gastric cancer, thyroid cancer, lung cancer, leukemia, pancreatic cancer, melanoma, brain cancer, kidney cancer, prostate cancer, ovarian cancer, or breast cancer.

In some embodiments, the lung cancer is non-small cell lung cancer carrying the EGFR^{L858R/T790M/C797S} or EGFR^{Δ19del/T790M/C797S} mutation.

A method for inhibiting mutated EGFR in patients, comprising administering a crystal form of the compound shown in formula I or a composition containing a crystal form of the compound shown in formula I in a therapeutically effective amount to patients in need; further, the crystal form is selected from one or more of the above-mentioned crystal form α, crystal form β, crystal form γ and crystal form δ.

The use of a crystal form of the compound represented by formula I or a composition containing a therapeutically effective amount of a crystal form of the compound represented by formula I in the preparation of medicines; further, the crystal form is selected from the above-mentioned crystal form α, crystal form β, one or more of crystal form γ and crystal form δ.

In some embodiments, the medicament is used to treat or prevent cancer.

In some embodiments, the cancer is colon cancer, stomach cancer, thyroid cancer, lung cancer, leukemia, pancreatic cancer, melanoma, brain cancer, kidney cancer, prostate cancer, ovarian cancer, or breast cancer.

In some embodiments, the lung cancer is non-small cell lung cancer carrying the EGFR^{L858R/T790M/C797S} or EGFR^{Δ19del/T790M/C797S} mutation.

On the other hand, the present invention also provides a salt of the compound represented by formula I.

In some embodiments, a compound of Formula I forms the corresponding salt with an acid. These salts can exist in various physical forms. For example, it can be in solution, suspension or solid form. In certain embodiments, the salt is in solid form. In solid form, the salt may be amorphous, crystalline or mixtures thereof.

Specifically, the salt of the compound represented by formula I is malate, hydrochloride, phosphate, tartrate, fumarate, succinate or methanesulfonate of the compound represented by formula I.

The malate salts of the compounds represented by formula I are exemplified below.

In some embodiments, the malate salt is L-malate.

In some embodiments, L-malate has the structure of a compound of Formula II:

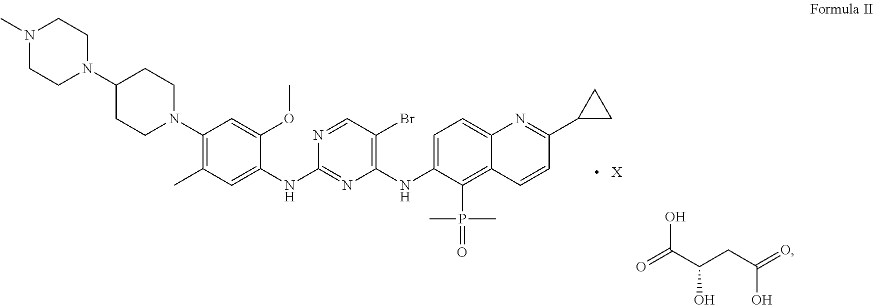

wherein, x is selected from 0.5-5.

In some embodiments, x is selected from 0.5-3.0, further 0.8-3.0; further 1.0, 2.0 or 3.0.

In some embodiments, x is selected from 0.5, 0.8, 1.0, 1.2, 1.5, 1.8, 2.0, 2.2, 2.5, 2.8, 3.0, 3.2, 3.5, 3.8, 4.0, 4.2, 4.5, 4.8, 5.0 or any other value in the range of 0.5-5 The present invention provides solid forms of compounds of formula II.

In some embodiments, the solid form is selected from amorphous or crystalline forms.

In some embodiments, the compound represented by Formula II is selected from the following compounds represented by Formula III:

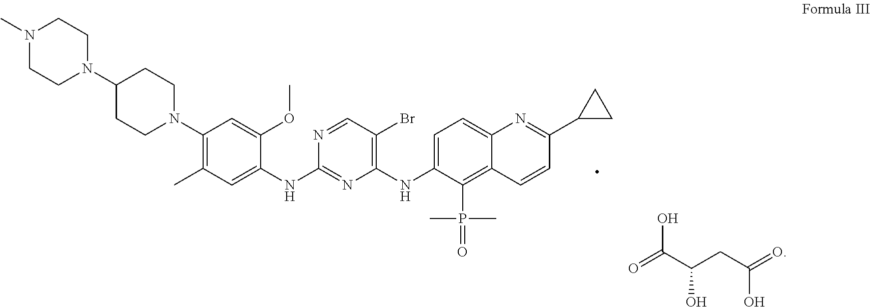 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1 ; O=C(O)C[C@H](O)C(=O)O]

The present invention provides solid forms of compounds of formula III.

In some embodiments, the solid form is selected from amorphous or crystalline forms.

In some embodiments, the crystalline form of said compound shown in Formula III is selected from any one or more of crystalline form A, crystalline form B, crystalline form C, crystalline form D, crystalline form E, crystalline form F, crystalline form G, crystalline form H, crystalline form I, and crystalline form J.

In some embodiments, the crystalline form A is characterized by having an X-ray powder diffraction pattern comprising characteristic peak in term of angle 2θ at 5.5±0.2°, 8.3±0.2°, 15.1±0.2° and 17.9±0.2°; further, the X-ray powder diffraction pattern of crystalline form A comprises one or more of the following diffraction angles 2θ: 7.8±0.2°, 9.2±0.2°, 11.3±0.2°, 11.7±0.2°, 13.6±0.2°, 13.8±0.2°, 16.4±0.2°, 16.6±0.2°, 17.2±0.2°, 20.1±0.2°, 20.9±0.2°; further, comprising characteristic peaks of 5.5±0.2°, 8.3±0.2°, 13.8±0.2°, 15.1±0.2°, 16.6±0.2° and 17.9±0.2°; furthermore, comprising characteristic peaks of 5.5±0.2°, 8.3±0.2°, 13.6±0.2°, 13.8±0.2°, 15.1±0.2°, 16.6±0.2° and 17.9±0.2°; furthermore, comprising characteristic peaks of 5.5±0.2°, 7.8±0.2°, 8.3±0.2°, 9.2 0.2°, 11.3±0.2°, 11.7±0.2°, 13.6±0.2°, 13.8±0.2°, 15.1±0.2°, 16.4±0.2°, 16.6±0.2°, 17.2±0.2°, 17.9±0.2°, 20.1±0.2°, 20.9±0.2°; furthermore, the X-ray powder diffraction pattern of the crystal form A is basically shown in FIG. 5.

In some embodiments, the crystal form A is a hydrate.

In some embodiments, the crystal form A is substantially pure, and its crystal form purity is ≥85%; further, the crystal form purity is ≥95%; further, the crystal form purity is ≥99%; further, the purity of the crystal form is ≥99.5%.

In some embodiments, the crystal form A is a hydrate crystal form; further, the crystal form A contains y molar equivalents of water, and the y is selected from 0.5 to 4.0; further, the y is selected from 0.5, 0.8, 1.0, 1.2, 1.5, 1.8, 2.0, 2.2, 2.5, 2.8, 3.0, 3.2, 3.5, 3.8 or 4.0.

In some embodiments, the y is selected from 0.5-2.5; further, the y is selected from 1.0-2.5.

In some embodiments, the y is selected from 0.5-2.0; further, the y is selected from 1.0-2.0. Furthermore, y is 1.0.

In some embodiments, the moisture content contained in the crystal form A of the compound represented by formula III is 1% to 5%; further, the moisture content contained in the crystal form A of the compound represented by formula III is 1%-4%; further specifically, the moisture content contained in the crystal form A of the compound represented by formula III is 1.0%-3.70%; further, the moisture content contained in the crystal form A of the compound represented by formula III is 2.0%-3.7%.

In some embodiments, the crystalline form B is characterized by having an X-ray powder diffraction pattern comprising characteristic peak in term of angle 2θ at 5.6±0.2°, 10.0 0.2°, 11.1±0.2°, 13.0±0.2°, 13.7±0.2°, 14.4±0.2°, 18.0±0.2°, 19.0±0.2° 20.2±0.2°, and 20.6±0.2°; further, the X-ray powder diffraction spectrum of the crystal form B is basically as shown in FIG. 6.

In some embodiments, the crystal form B is substantially pure, and its crystal form purity is ≥85%; further, the crystal form purity is ≥95%; further, the crystal form purity is ≥99%; further, the purity of the crystal form is ≥99.5%.

In some embodiments, the crystal form B is a hydrate crystalline form.

In some embodiments, the crystalline form C is characterized by having an X-ray powder diffraction pattern comprising characteristic peak in term of angle 2θ at 7.2±0.2°, 8.4±0.2°, 9.2±0.2°, 11.6±0.2°, 12.3±0.2°, 14.2±0.2°, 16.8±0.2°, 18.0±0.2°, and 20.6±0.2°; further, the X-ray powder diffraction pattern of the crystal form C is basically shown in FIG. 7.

In some embodiments, the crystal form C is substantially pure, and its crystal form purity is ≥85%; further, the crystal form purity is ≥95%; further, the crystal form purity is ≥99%; further, the purity of the crystal form is ≥99.5%.

In some embodiments, the crystal form C is an anhydrous form.

In some embodiments, the crystalline form D is characterized by having an X-ray powder diffraction pattern comprising characteristic peak in term of angle 2θ at 5.4±0.2°, 8.3±0.2°, 14.8±0.2°, 16.4±0.2°, and 17.6±0.2°; further, the X-ray powder diffraction pattern of the crystal form D is basically as shown in FIG. 8.

In some embodiments, the crystalline form D is substantially pure, and its crystalline purity is ≥85%; further, the crystalline purity is ≥95%; further, the crystalline purity is ≥99%; further, the purity of the crystal form is ≥99.5%.

In some embodiments, the crystalline form D is an anhydrous form.

In some embodiments, the crystalline form E is characterized by having an X-ray powder diffraction pattern comprising characteristic peak in term of angle 2θ at 7.1±0.2°, 11.9±0.2°, 14.3±0.2°, 15.1±0.2°, 15.9±0.2°, 19.3±0.2° and 20.5±0.2°; further, the X-ray powder diffraction pattern of the crystal form E is basically shown in FIG. 9.

In some embodiments, the crystalline form E is substantially pure, and its crystalline purity is ≥85%; further, the crystalline purity is ≥95%; further, the crystalline purity is ≥99%; further, the purity of the crystal form is ≥99.5%.

In some embodiments, the crystalline form E is an anhydrous form.

In some embodiments, the crystalline form F is characterized by having an X-ray powder diffraction pattern comprising characteristic peak in term of angle 2θ at 6.6±0.2°, 7.4±0.2°, 10.5±0.2°, 16.4±0.2°, and 21.1±0.2°; further, the X-ray powder diffraction pattern of the crystal form F is basically as shown in FIG. 10.

In some embodiments, the crystalline form F is substantially pure, and its crystalline purity is ≥85%; further, the crystalline purity is ≥95%; further, the crystalline purity is ≥99%; further, the purity of the crystal form is ≥99.5%.

In some embodiments, the crystalline form F is a tetrahydrofuran solvate crystalline form.

In some embodiments, the crystalline form G is characterized by having an X-ray powder diffraction pattern comprising characteristic peak in term of angle 2θ at 5.0±0.2°, 10.0±0.2°, 15.0±0.2°, and 19.5±0.2°; further, the X-ray powder diffraction pattern of the crystalline form G is basically shown in FIG. 11.

In some embodiments, the crystal form G is substantially pure, and its crystal form purity is ≥85%; further, the crystal form purity is ≥95%; further, the crystal form purity is ≥99%; further, the purity of the crystal form is ≥99.5%.

In some embodiments, the crystal form G is an anhydrous form.

In some embodiments, the crystalline form H is characterized by having an X-ray powder diffraction pattern comprising characteristic peak in term of angle 2θ at 4.7±0.2°, 9.3±0.2°, and 14.0±0.2°; further, the X-ray powder diffraction pattern of the crystalline form G is basically shown in FIG. 12.

In some embodiments, the crystalline form H is substantially pure, and its crystalline purity is ≥85%; further, the crystalline purity is ≥95%; further, the crystalline purity is ≥99%; further, the purity of the crystal form is ≥99.5%.

In some embodiments, the crystalline form H is an ethanol solvate crystalline form.

In some embodiments, the X-ray powder diffraction pattern of Form I is substantially as shown in FIG. 13.

In some embodiments, the crystalline form I is substantially pure, and its crystalline purity is ≥85%; further, the crystalline purity is ≥95%; further, the crystalline purity is ≥99%; further, the purity of the crystal form is ≥99.5%.

In some embodiments, the crystalline form I is a hydrate crystalline form.

In some embodiments, the crystalline form J is characterized by having an X-ray powder diffraction pattern comprising characteristic peak in term of angle 2θ at 9.0±0.2°, 11.2±0.2°, 11.7±0.2°, 12.2±0.2°, 14.0±0.2°, 15.5±0.2°, 16.2±0.2°, 18.0±0.2° 19.2±0.2°, and 20.0±0.2°; furthermore, the X-ray powder diffraction spectrum of the crystal form J is essentially shown in FIG. 14.

In some embodiments, the crystal form J is substantially pure, and its crystal form purity is ≥85%; further, the crystal form purity is ≥95%; further, the crystal form purity is ≥99%; further, the purity of the crystal form is ≥99.5%.

In some embodiments, the Form J is an anhydrous form.

In some embodiments, x in the compound represented by Formula II is selected from 2.0, and its structure is represented by Formula IV:

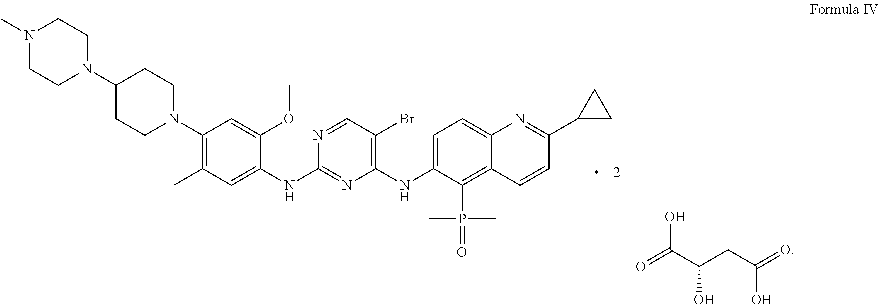 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1 ; "2" ; O=C(O)C[C@H](O)C(=O)O]

The present invention provides solid forms of compounds of formula IV.

In some embodiments, the solid form is selected from amorphous or crystalline forms.

In some embodiments, the crystal form of the compound represented by Formula IV is selected from one or more of crystal form A, crystal form B, and crystal form C.

In some embodiments, the crystalline form A is characterized by having an X-ray powder diffraction pattern comprising characteristic peak in term of angle 2θ at 5.5±0.2°, 6.2±0.2°, 6.5±0.2°, 9.1±0.2°, 9.4±0.2°, 11.2±0.2°, 13.1±0.2°, 13.4±0.2°, 15.1±0.2°, 18.0±0.2°, 18.2±0.2°, 19.5±0.2°, 20.4±0.2°, 21.2±0.2°, 21.3±0.2°, 21.7±0.2°, 23.3±0.2°, 24.9±0.2°; furthermore, the X-ray powder diffraction spectrum of crystal form A is essentially shown in FIG. 16.

In some embodiments, the crystal form A is substantially pure, and its crystal form purity is ≥85%; further, the crystal form purity is ≥95%; further, the crystal form purity is ≥99%; further, the purity of the crystal form is ≥99.5%.

In some embodiments, the crystalline form B is characterized by having an X-ray powder diffraction pattern comprising characteristic peak in term of angle 2θ at 7.6±0.2°, 9.8±0.2°, 11.6±0.2°, 19.1±0.2°, 19.5±0.2°, 19.8±0.2°, 21.3±0.2°, 22.2±0.2°, 23.1±0.2°; furthermore, the X-ray powder diffraction spectrum of crystal form B is essentially shown in FIG. 17.

In some embodiments, the crystal form B is substantially pure, and its crystal form purity is ≥85%; further, the crystal form purity is ≥95%; further, the crystal form purity is ≥99%; further, the purity of the crystal form is ≥99.5%.

In some embodiments, the crystalline form C is characterized by having an X-ray powder diffraction pattern comprising characteristic peak in term of angle 2θ at 8.0±0.2°, 8.7±0.2°, 12.3±0.2°, and 21.9±0.2°; further, the X-ray powder diffraction pattern of Form C is essentially shown in FIG. 18.

In some embodiments, the crystal form C is substantially pure, and its crystal form purity is ≥85%; further, the crystal form purity is ≥95%; further, the crystal form purity is ≥99%; further, the purity of the crystal form is ≥99.5%.

In some embodiments, x in the compound represented by Formula II is selected from 3.0, and its structure is represented by Formula V:

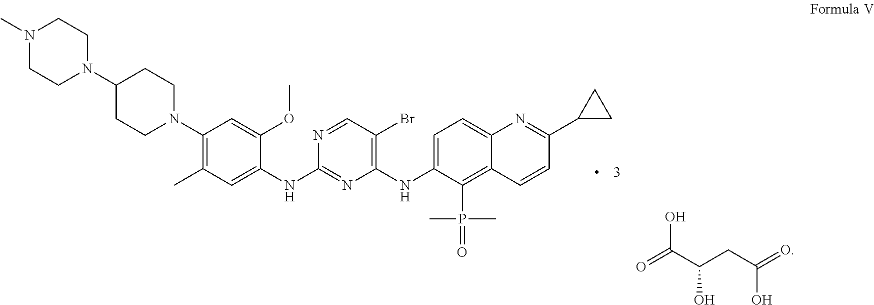 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1 ; "3" ; O=C(O)C[C@H](O)C(=O)O]

The present invention provides solid forms of compounds represented by formula V.

In some embodiments, the solid form is selected from amorphous or crystalline forms.

In some embodiments, the crystal form of the compound represented by Formula V is Form A.

In some embodiments, the crystalline form A is characterized by having an X-ray powder diffraction pattern comprising characteristic peak in term of angle 2θ at 6.4±0.2°, 7.4±0.2°, 9.7±0.2°, 11.4±0.2°, 12.7±0.2°, 16.7±0.2°, 18.0 0.2°, 19.0±0.2°, 20.5±0.2°, 21.0±0.2°, 22.2±0.2°, 23.0±0.2°; further, the X-ray powder diffraction spectrum of the crystal form A is substantially as shown in FIG. 19.

In some embodiments, the crystal form A is substantially pure, and its crystal form purity is ≥85%; further, the crystal form purity is ≥95%; further, the crystal form purity is ≥99%; further, the purity of the crystal form is ≥99.5%.

The hydrochloride salts of the compounds represented by formula I are exemplified below.

In some embodiments, in the hydrochloride salt of the compound represented by Formula I, the molar ratio of the compound represented by Formula I to hydrochloric acid is 1:1.

The present invention provides a solid form of the hydrochloride salt of the compound represented by formula I.

In some embodiments, the solid form is selected from amorphous or crystalline forms.

In some embodiments, the hydrochloride crystal form of the compound represented by Formula I is selected from one or more of crystal form A and crystal form B.

In some embodiments, the crystalline form A is characterized by having an X-ray powder diffraction pattern comprising characteristic peak in term of angle 2θ at 6.0±0.2°, 7.4±0.2°, 11.0±0.2°, 13.8±0.2°, 14.2±0.2°, 16.1±0.2°, 18.1±0.2°, 18.5±0.2°, 20.1±0.2°, 21.4±0.2°, 23.1±0.2°, 23.9±0.2°, 24.0±0.2°, 25.6±0.2°; further, The X-ray powder diffraction pattern of the crystalline form A is basically shown in FIG. 15.

In some embodiments, the crystal form A is substantially pure, and its crystal form purity is ≥85%; further, the crystal form purity is ≥95%; further, the crystal form purity is ≥99%; further, the purity of the crystal form is ≥99.5%.

In some embodiments, the crystalline form B is characterized by having an X-ray powder diffraction pattern comprising characteristic peak in term of angle 2θ at 6.6±0.2°, 7.1±0.2°, 9.2±0.2°, 11.4±0.2°, 12.5±0.2°, 13.1±of 0.2°, 19.3±0.2°, 23.7±0.2°, 24.0±0.2°, and 26.5±0.2°; further, the X-ray powder diffraction spectrum of the crystal form B is basically as shown in FIG. 20.

In some embodiments, the crystal form B is substantially pure, and its crystal form purity is ≥85%; further, the crystal form purity is ≥95%; further, the crystal form purity is ≥99%; further, the purity of the crystal form is ≥99.5%.

In some embodiments, the Form B is a hydrate crystalline form.

Tartrate salts of compounds represented by formula I are exemplified below.

In some embodiments, the tartrate is L-tartrate.

The present invention provides a solid form of the L-tartrate salt of the compound represented by formula I.

In some embodiments, the solid form is selected from amorphous or crystalline forms.

In some embodiments, the L-tartrate crystal form of the compound represented by Formula I is Form A.

In some embodiments, the crystalline form A is characterized by having an X-ray powder diffraction pattern comprising characteristic peak in term of angle 2θ at 5.8±0.2°, 7.0 0.2°, 9.9±0.2°, 11.7±0.2°, 12.6±0.2°, 14.0±0.2°, 17.8±0.2°, and 18.9±0.2°; further, the X-ray powder diffraction pattern of the crystal form A is basically shown in FIG. 21.

In some embodiments, the crystal form A is substantially pure, and its crystal form purity is ≥85%; further, the crystal form purity is ≥95%; further, the crystal form purity is ≥99%; further, the purity of the crystal form is ≥99.5%.

In some embodiments, the Form A is a hydrate crystalline form.

The fumarate salts of the compounds represented by formula I are exemplified below.

The present invention provides a solid form of the fumarate salt of the compound represented by formula I.

In some embodiments, the solid form is selected from amorphous or crystalline forms.

In some embodiments, the fumarate crystal form of the compound represented by Formula I is Form B.

In some embodiments, the crystalline form B is characterized by having an X-ray powder diffraction pattern comprising characteristic peak in term of angle 2θ at 7.2±0.2°, 8.1±0.2°, 8.4±0.2°, 9.2 0.2°, 14.3±0.2°, 17.0±0.2°, 18.1±0.2°, and 20.7±0.2°; further, the X-ray powder diffraction pattern of the crystal form B is basically as shown in FIG. 22.

In some embodiments, the crystal form B is substantially pure, and its crystal form purity is ≥85%; further, the crystal form purity is ≥95%; further, the crystal form purity is ≥99%; further, the purity of the crystal form is ≥99.5%.

In some embodiments, the crystal form B is a solvate crystal form; further, it is an acetone solvate crystal form.

The succinate salts of the compounds represented by formula I are exemplified below.

The present invention provides a solid form of the succinate salt of the compound represented by formula I.

In some embodiments, the solid form is selected from amorphous or crystalline forms.

In some embodiments, the succinate crystal form of the compound represented by Formula I is Form A.

In some embodiments, the crystalline form A is characterized by having an X-ray powder diffraction pattern comprising characteristic peak in term of angle 2θ at 7.2±0.2°, 8.0 0.2°, 8.4±0.2°, 9.1±0.2°, 11.7±0.2°, 12.4±0.2°, 14.1±0.2°, 16.8±0.2°, 18.1±0.2°, and 20.6±0.2°; further, the X-ray powder diffraction spectrum of the crystal form A is basically as shown in FIG. 23.

In some embodiments, the crystal form A is substantially pure, and its crystal form purity is ≥85%; further, the crystal form purity is ≥95%; further, the crystal form purity is ≥99%; further, the purity of the crystal form is ≥99.5%.

In some embodiments, the Form A is an anhydrous form.

Methanesulfonate salts of compounds represented by formula I are exemplified below.

The present invention provides a solid form of the mesylate salt of the compound represented by formula I.

In some embodiments, the solid form is selected from amorphous or crystalline forms.

In some embodiments, the mesylate crystal form of the compound represented by Formula I is Form A.

In some embodiments, the crystalline form A is characterized by having an X-ray powder diffraction pattern comprising characteristic peak in term of angle 2θ at 7.3±0.2°, 10.5±0.2°, 15.1±0.2°, 15.5±0.2°, 20.9±0.2°, 21.4±0.2° and 22.2±0.2°; further, the X-ray powder diffraction pattern of the crystal form A is basically shown in FIG. 24.

In some embodiments, the crystal form A is substantially pure, and its crystal form purity is ≥85%; further, the crystal form purity is ≥95%; further, the crystal form purity is ≥99%; further, the purity of the crystal form is ≥99.5%.

In some embodiments, the crystal form A is a solvate crystal form; further, it is an acetonitrile solvate crystal form.

The phosphate salts of the compounds represented by formula I are exemplarily listed below.

The present invention provides solid forms of phosphate salts of compounds represented by Formula I.

In some embodiments, the solid form is selected from amorphous or crystalline forms.

In some embodiments, the phosphate crystal form of the compound represented by Formula I is Form D.

In some embodiments, the crystalline form D is characterized by having an X-ray powder diffraction pattern comprising characteristic peak in term of angle 2θ at 5.9±0.2°, 7.0 0.2°, 10.3±0.2°, 11.0 0.2°, 12.2±0.2°, 13.8±0.2°, 14.1±0.2°, 16.6±0.2°, 17.6±0.2°, 18.9±0.2°, 19.2±0.2°, 19.7±0.2°, 20.3±0.2°, 20.6±0.2°, 22.6±0.2°, 23.1±0.2°; further, the X-ray powder diffraction pattern of the crystal form D is basically shown in FIG. 25.

In some embodiments, the crystalline form D is substantially pure, and its crystalline purity is ≥85%; further, the crystalline purity is ≥95%; further, the crystalline purity is ≥99%; further, the purity of the crystal form is ≥99.5%. In some embodiments, the Form D is a hydrate crystalline form.

A composition comprising a therapeutically effective amount of a salt of a compound represented by Formula I.

In some embodiments, the composition further includes pharmaceutically acceptable excipients.

A method for inhibiting various forms of EGFR mutations, including one or more of L858R, Δ19del, T790M and C797S mutations, the method comprising administering to a patient a salt of a compound represented by Formula I or a therapeutically effective amount of A composition of salts of the compound represented by formula I.

A method for treating EGFR driven cancer, comprising administering a salt of a compound of formula I in a therapeutically effective amount to a patient in need, or a composition comprising a salt of a compound of formula I in a therapeutically effective amount.

In some embodiments, EGFR driven cancers are characterized by the presence of one or more mutations selected from: (i) C797S, (ii) L858R and C797S, (iii) C797S and T790M, (iv) L858R, T790M, and C797S, (v) Δ19del, T790M and C797S, (vi) Δ19del and C797S, (vii) L858R and T790M, or (viii) Δ19del and T790M.

In some embodiments, EGFR driven cancers are colon cancer, gastric cancer, thyroid cancer, lung cancer, leukemia, pancreatic cancer, melanoma, brain cancer, kidney cancer, prostate cancer, ovarian cancer, or breast cancer.

In some embodiments, the lung cancer is non-small cell lung cancer carrying the EGFR L858R/T790M/C797S or EGFR Δ19del/T790M/C797S mutation.

A method for inhibiting mutated EGFR in patients, comprising administering a therapeutic effective amount of salt of a compound shown in formula I or a composition containing a therapeutic effective amount of salt of a compound shown in formula I to patients in need.

The use of a salt of a compound shown in formula I or a composition containing a therapeutically effective amount of compound salt shown in formula I in the preparation of drugs.

In some embodiments, the drug is used for treating or preventing cancer.

In some embodiments, the cancer is colon cancer, stomach cancer, thyroid cancer, lung cancer, leukemia, pancreatic cancer, melanoma, brain cancer, kidney cancer, prostate cancer, ovarian cancer, or breast cancer.

In some embodiments, the lung cancer is non-small cell lung cancer carrying the EGFR^{L858R/T790M/C797S} or EGFR^{Δ19del/T790M/C797S} mutation.

Further, the salt of the compound shown in formula I can be selected from all the aforementioned salts and crystal types thereof, such as the crystal form of the salt of the compound shown in formula I; selected from the salt shown in formula II Compound; selected from the compound represented by formula III; selected from the crystal form of the compound represented by formula III; selected from the compound represented by formula III, crystal form A, crystal form B, crystal form C, crystal form D, crystal form E, crystal form F, one or more of crystal form G, crystal form H, crystal form I, and crystal form J.

### Technical Effect

All compounds of the present invention, including the crystal form, salt and crystal form of the compound represented by Formula I, have good pharmaceutical properties, such as high Cₘₐₓ and high exposure. Among them, the crystal form possesses good stability. properties, such as good light, high temperature, high humidity stability and the like, therefore obtain good medicinal properties.

### DEFINITION AND DESCRIPTION

Unless otherwise stated, the following terms and phrases used herein are intended to contain the following meanings. A particular phrase or term should not be considered uncertain or unclear in the absence of a specific definition, but should be understood in its ordinary meaning. Where trade names appear herein, they are intended to refer to their corresponding commodity or their active ingredients.

As described herein, the new crystalline form can be identified by X-ray powder diffraction spectra. However, those skilled in the art know that the peak intensity and/or peak situation of X-ray powder diffraction may vary due to different experimental conditions, such as different diffraction test conditions and/or orientation priorities. At the same time, due to the different accuracy of different instruments, the measured diffraction angle 2θ will exist an error of approximately ±0.2°. However, it is known that the relative intensity value of a peak is more dependent on certain properties of the measured sample than the position of the peak, such as the size of the crystals in the sample, the orientation of the crystals and the purity of the material being analyzed, therefore the peaks displayed Intensity deviations of approximately ±20% or greater are possible. However, despite the existence of experimental errors, instrument errors, orientation preferences, and the like, those skilled in the art can obtain sufficient information for identifying crystal forms from the XRPD data provided in this patent.

In the present invention, “having an X-ray powder diffraction pattern essentially as shown in FIG. 1” or “having an X-ray powder diffraction pattern essentially as shown in FIG. 2” refers to the main peaks are as shown in FIG. 1 or FIG. 2, wherein the main peak refers to a relative intensity value exceeding 10%, preferably exceeding 30%, compared with the highest peak in FIG. 1 or FIG. 2 (whose relative intensity is designated as 100%).

The “crystalline form” of the present invention can be present in the sample at 0.0001%-100%. Therefore, as long as the sample contains even trace amounts such as greater than 0.0001%, greater than 0.001% or greater than 0.01% of the “crystalline form” of the present invention. The “crystal forms” should be understood as falling within the protection scope of the present invention. In order to describe the various parameters of the “crystal form” described in the present invention more clearly, the present invention tests various parameters on samples containing a basically pure “crystal form”, and characterizes and identifies the crystal form. The term “substantially pure” means that the sample consists essentially of one major crystalline form and is substantially free of one or more other crystalline forms or amorphous forms, and the purity of the major crystalline form is at least 80%, or at least 85%, or at least 90%, or at least 93%, or at least 95%, or at least 98%, or at least 99%.

As used herein, the terms “crystal form,” “crystalline form,” “form” and related terms are used interchangeably and refer to crystalline solid forms unless otherwise indicated. Crystal forms include single-component crystal forms and multi-component crystal forms, including but not limited to solvent-free forms (such as anhydrous forms), solvates, hydrates, co-crystals and other molecular complexes and their polymorphs, as well as salts, salt solvates, salt hydrates, salt co-crystals, other molecular complexes of salts and their polymorphs. In some embodiments, the crystalline form of a substance may be substantially free of amorphous and/or other crystalline forms. In certain embodiments, crystalline forms of a substance may contain less than about 50% by weight of one or more amorphous and/or other crystalline forms. In some embodiments, crystalline forms of substances may be physically and/or chemically pure.

If not otherwise stated, the term “solvate” as used herein refers to a molecular complex including a drug substance and a stoichiometric or non-stoichiometric amount of solvent molecules. The drug substance may be a free base or a pharmaceutically acceptable form thereof, Salt, eutectic, eutectic of salts or other molecular complexes. When the solvent is water, the solvate is a “hydrate”.

Hydrate forms can be stoichiometric hydrates in which water is present in the crystal lattice in defined molar equivalents, independent of humidity, such as hemihydrate, monohydrate, dihydrate and the like. Hydrate forms can also be non-stoichiometric hydrates, also known as variable hydrates, wherein the water content is variable and dependent on external conditions such as humidity, temperature, drying conditions, and the like. Therefore other hydrate forms such as channel hydrates are also included within the meaning of this term.

Unless otherwise stated, the term “anhydrous” as used herein refers to an anhydrous, solvent-free crystalline form.

If not otherwise stated, the term “amorphous” as used herein refers to a disordered solid form of molecules and/or ions, which is not crystalline. Amorphous does not show a well-defined X-ray diffraction pattern with sharp well-defined peaks. Compounds are intended to cover any single solid form of the free base, or mixtures of solid forms, unless otherwise specified.

Polymorphs of compounds can be obtained by a number of methods known in the art. Such methods include, but are not limited to, melt recrystallization, melt cooling, solvent recrystallization, desolvation, rapid evaporation, rapid cooling, slow cooling, vapor diffusion and sublimation.

In the present invention, As used herein, the term “therapeutically effective amount” refers to the amount of a compound/crystalline form that is sufficient to affect the treatment of a disease, disorder or symptom when administered to a subject for the treatment of a disease, or at least one clinical symptom of a disease or condition. The “therapeutically effective amount” may vary with the compound, the disease, the symptom, the severity of the symptom, the age of the patient being treated, and/or the weight of the patient being treated. In any particular case, a suitable amount may be apparent to those of skill in the art or may be determined using routine experimentation. In the case of combination therapy, “therapeutically effective amount” refers to the total amount of the combination subject that effectively treats the disease or condition.

All dosage forms of the pharmaceutical composition of the present invention can be prepared by conventional methods in the pharmaceutical field. For example, the active ingredient is mixed with one or more excipients and then formulated into the desired dosage form.

“Pharmaceutically acceptable excipients” refer to conventional pharmaceutical excipients suitable for desired pharmaceutical preparations, for example: diluents and excipients such as water, various organic solvents; fillers such as starch, sucrose; binders such as cellulose derivatives, alginates, gelatin and polyvinylpyrrolidone (PVP); humectants such as glycerin; disintegrants such as agar, calcium carbonate and sodium bicarbonate; absorption enhancers such as quaternary ammonium compounds; surfactants such as cetyl alcohol; absorbent carriers such as kaolin and bentonite; lubricants such as talc, calcium stearate, magnesium stearate and polyethylene glycol.

In addition, other pharmaceutically acceptable excipients, such as dispersants, stabilizers, thickeners, complexing agents, buffers, penetration enhancers, polymers, fragrances, sweeteners and dyes, can also be added to the pharmaceutical composition. Preference is given to the use of excipients suitable for the desired dosage form and intended mode of administration.

The terms “disease,” “disorder,” or “condition” refer to any disease, ailment, disease, symptom, or indication.

The term “multiple” means two or more, for example, “multiple” means “two or more”, and “multiple” means “two or more”.

Unless otherwise specified, the compounds of the present invention include various types such as free bases, salts, crystal forms, and solvates. The solvate refers to solvent molecules that participate in the formation of the crystal lattice of compound molecules, such as hydrates, tetrahydrofuran solvates, methanol solvates, ethanol solvates, and the like.

It should be noted that for the same crystal form, the position of the endothermic peak of DSC may differ depending on factors such as measuring instruments, measuring methods/conditions. There may be an error in the position of the endothermic peak for any particular crystal form, the error can be ±10° C. (for example the error can be ±9° C., ±8° C., ±6° C., ±5° C., ±4° C., ±3° C., ±2° C., ±1° C., ±0.5° C.). Therefore, this error should be taken into account when determining each crystal form, and it is within the scope of the present invention.

It should be noted that for the same crystal form, the position of the weight loss temperature of TGA may differ depending on factors such as measuring instruments, measuring methods/conditions. There may be an error in the location of the weight loss temperature for any particular crystal form, the error can be ±10° C. (for example the error can be ±9° C., ±8° C., ±6° C., ±5° C., ±4° C., ±3° C., ±2° C., ±1° C., ±0.5° C.). Therefore, this error should be taken into account when determining each crystal form, and it is within the scope of the present invention.

### Instruments and Analytical Methods

X-ray Powder diffractometer, XRPD

| | |
|---|---|
| Equipment Name | X-ray Powder diffractometer |
| Instrument | Bruker D8 Advance |
| Technical | Copper target wavelength is |
| Indicators | 1.54 Å Kα radiation (40 kV, |
| | 40 mA), θ-2θ goniometer, |
| | Ni monochromator, Lynxeye detector |

| | | |
|---|---|---|
| Test | Detection angle | 3-40°2θ |
| Parameters | Step length | 0.02°2θ |
| | Speed | 0.2-0.6 s · step⁻¹ |

Those skilled in the art can understand that in the process of obtaining XRPD spectra, in order to reduce errors, relevant data can be subjected to appropriate scientific processing, such as baseline correction processing. Those skilled in the art can also understand that when operated under different laboratory conditions, there will be slight differences in the diffraction angle 2θ or resolution of the XRPD spectrum obtained. It should be understood that the XRPD spectra of the crystal form of the compound represented by Formula I and the crystal form of the compound salt represented by Formula I provided by the present invention are not limited to the X-ray powder diffraction patterns shown in the accompanying drawings, which are basically the same as those shown in the accompanying drawings. The crystals having X-ray powder diffraction patterns fall within the scope of the present invention.

### DESCRIPTION OF FIGURE

FIG. 1: XRPD pattern of crystalline form α of the compound represented by formula I.

FIG. 2: XRPD pattern of crystal form β of the compound represented by formula I.

FIG. 3: XRPD pattern of crystal form γ of the compound represented by formula I.

FIG. 4: XRPD pattern of crystal form δ of the compound represented by formula I.

FIG. 5: XRPD pattern of crystal form A of the compound represented by formula III.

FIG. 5-1: DSC spectrum of crystal form A of the compound represented by formula III.

FIG. 5-2: DVS spectrum of crystal form A of the compound represented by formula III.

FIG. 5-3: Ellipsoid diagram of the single crystal molecular three-dimensional structure of Form A of the compound represented by Formula III.

FIG. 6: XRPD pattern of crystal form B of the compound represented by formula III.

FIG. 7: XRPD pattern of crystal form C of the compound represented by formula III.

FIG. 8: XRPD pattern of crystal form D of the compound represented by formula III.

FIG. 9: XRPD pattern of crystal form E of the compound represented by formula III.

FIG. 10: XRPD pattern of crystal form F of the compound represented by formula III.

FIG. 11: XRPD pattern of crystalline form G of the compound represented by formula III.

FIG. 12: XRPD pattern of crystal form H of the compound represented by formula III.

FIG. 13: XRPD pattern of crystal form I of the compound represented by formula III.

FIG. 14: XRPD pattern of crystal form J of the compound represented by formula III.

FIG. 15: XRPD pattern of hydrochloride form A of the compound represented by formula I.

FIG. 16: XRPD pattern of crystal form A of the compound represented by formula IV.

FIG. 17: XRPD pattern of crystal form B of the compound represented by formula IV.

FIG. 18: XRPD pattern of crystal form C of the compound represented by formula IV.

FIG. 19: XRPD pattern of crystal form A of the compound represented by formula V.

FIG. 20: XRPD pattern of hydrochloride crystal form B of the compound represented by formula I.

FIG. 21: XRPD pattern of L-tartrate crystal form A of the compound represented by formula I.

FIG. 22: XRPD pattern of fumarate crystal form B of the compound represented by formula I.

FIG. 23: XRPD pattern of the succinate crystal form A of the compound represented by formula I.

FIG. 24: XRPD pattern of crystal form A of the mesylate salt of the compound represented by formula I.

FIG. 25: XRPD pattern of phosphate crystal form D of the compound represented by formula I.

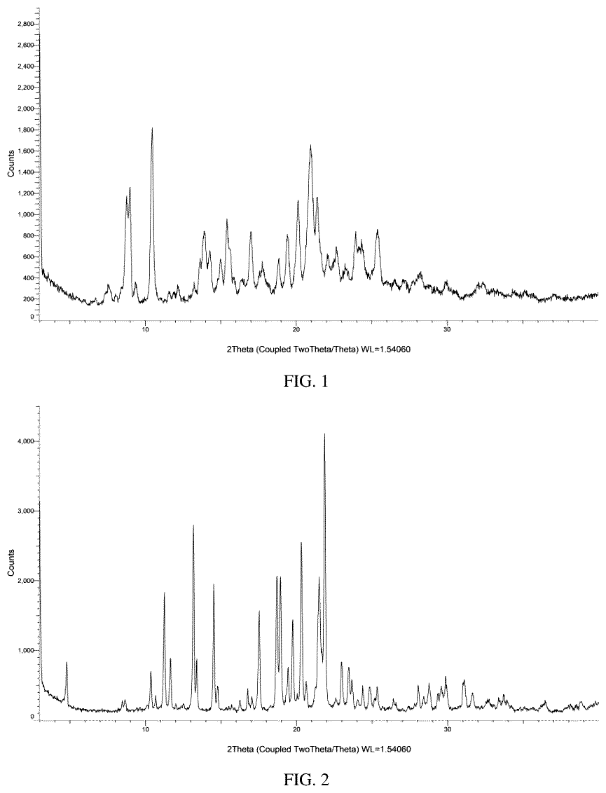

In the above FIGS. 1 to 25, the abscissa (X-axis) all represents the diffraction angle 2θ, and the unit is “°”; the ordinate (Y-axis) all represents the diffraction intensity, and the unit is “counts”.

### EXAMPLES

The present invention will be further illustrated by the examples given below, but the examples do not constitute any limitation on the scope of protection claimed by the present invention. In the specific embodiments of the present invention, unless otherwise specified, the techniques or methods described are conventional techniques or methods in the art. The solvent used in the present invention is commercially available, and the raw materials used are commercially available products unless otherwise specified.

### Abbreviation

AcOH: acetic acid;
DIEA: N,N-diisopropylethylamine;
DMF: N,N-dimethylformamide;
DMSO: dimethyl sulfoxide;
EA: ethyl acetate;
HEPES: 4-(2-hydroxyethyl)-1-piperazineethanesulfonic acid;
Xantphos: 4,5-bis(diphenylphosphine)-9,9-dimethylxanthene;
Pd(OAc)₂: palladium acetate
n-BuOH: n-butanol;
PTSA: p-toluenesulfonic acid;
PTLC: preparative thin layer chromatography;
LCMS: liquid chromatography-mass spectrometry;
h or hrs: hours;
Pd/C: palladium carbon;
MeOH: methanol;
NMP: N-methyl-2-pyrrolidone;
TLC: preparative thin layer chromatography;
Pd(dppf)Cl₂: 1,1′-bis(diphenylphosphine)ferrocene palladium dichloride;
Pd(PPh₃)₄: palladium tetrakistriphenylphosphine;
Pd-Ruphos G₃: methanesulfonic acid
(2-dicyclohexylphosphino-2′,6′-diisopropoxy-1,1′-biphenyl) (2-amino-1,1′-biphenyl-2-yl) palladium(II);
Ruphos: 2-dicyclohexylphosphine-2′,6′-diisopropoxybiphenyl;
Cs₂CO₃: cesium carbonate;
ACN: acetonitrile;
XRPD: X-ray powder diffraction.
DSC: Differential Scanning Calorimetry
DVS: dynamic vapor adsorption

### Example 1: Synthesis of the Compound Represented by Formula I

### (6-((5-bromo-2-((2-methoxy-5-methyl-4-(4-(4-methylpiperazin-1-yl))piperidin-1-yl)phenyl)amino)pyrimidin-4-yl)amino)-2-cyclopropylquinolin-5-yl)dimethylphosphine oxide

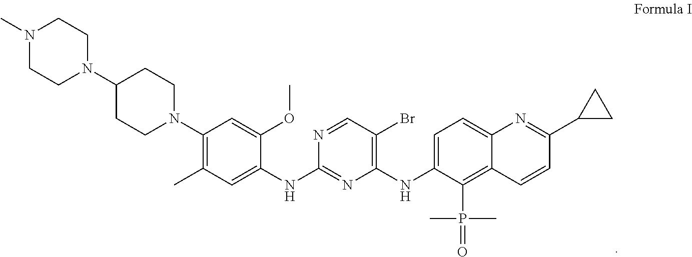 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1]

### Step 1: Synthesis of Compound 1-2

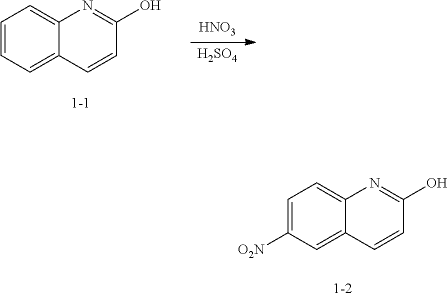

Compound 1-1 (25 g, 172.23 mmol) was dissolved in concentrated sulfuric acid (100 mL). Concentrated nitric acid (16.28 g, 175.67 mmol) was added dropwise at 0° C. After the addition was completed, the mixture was stirred at room temperature for 2 h. TLC monitors the complete reaction of raw materials. The reaction solution was slowly poured into 2 L of ice water to quench, and a light yellow solid precipitated. Stir for 1 h and filter. The filter cake was washed with 1 L of water. The filter cake was collected and dried to obtain compound 1-2 (24.5 g, 128.84 mmol), yield: 74.81%). MS: 191.04 [M+H]⁺

### Step 2: Synthesis of Compound 1-3

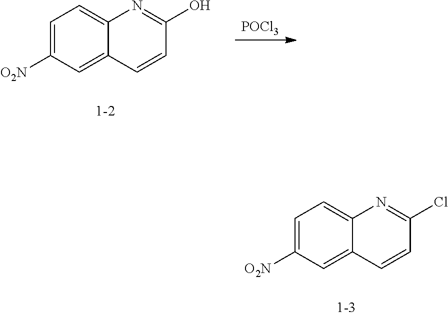

Compound 1-2 (24.5 g, 128.84 mmol) was dissolved in phosphine oxychloride (200 mL), heated to 100° C. and stirred overnight. LCMS monitors the complete reaction of raw materials. The reaction solution was lowered to room temperature, concentrated, and the residue was poured into 1 L of ice water, stirred for 0.5 h, filtered, and the filter cake was rinsed with 1 L of water. The filter cake was collected and dried to obtain compound 1-3 (25 g, 119.85 mmol, yield: 93.02%).

### Step 3: Synthesis of Compound 1-4

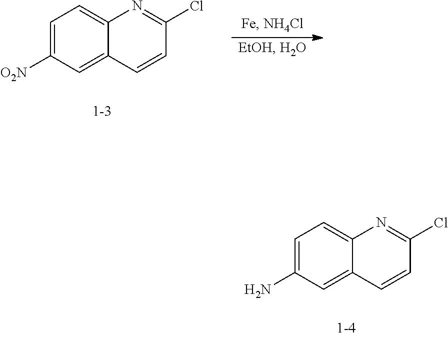

Dissolve compound 1-3 (25 g, 119.85 mmol) in 150 mL ethanol, add 30 mL H₂O, then add iron powder (33.47 g, 599.23 mmol), ammonium chloride (32.05 g, 599.23 mmol), and mix the reaction solution with heat to 90° C. and stir for 3 hours. The reaction solution was cooled to room temperature and filtered through diatomaceous earth. The filter cake was washed with ethanol several times. The filtrate was collected and concentrated. The residue was purified by Flash silica gel column (A: DCM, B: MeOH) to obtain compound 1-4 (16.9 g, 94.62 mmol, yield: 78.95%). MS: 179.03 [M+H]⁺

### Step 4: Synthesis of Compound 1-5

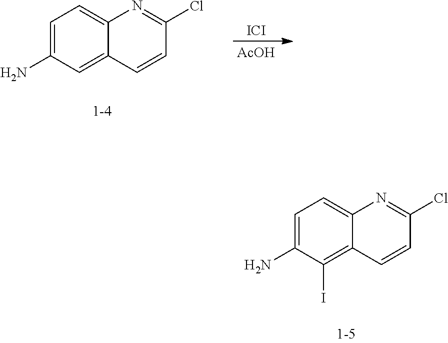

Compound 1-4 (16.9 g, 94.62 mmol) was dissolved in glacial acetic acid (320 mL), and solution of iodine chloride (18.43 g, 113.54 mmol) in acetic acid (80 mL) was added dropwise at room temperature. After addition, stirring was continued at room temperature for 2 h. LCMS monitors the complete reaction of raw materials. Add 500 mL n-hexane to the reaction solution to dilute it. If solid precipitates, filter it. Rinse the filter cake with n-hexane and drain it. Dissolve the filter cake in a mixed solvent of DCM: MeOH=10:1, wash it with saturated sodium carbonate solution twice, saturated sodium thiosulfate solution twice, and saturated sodium chloride once, dry, filter and concentrate. The residue was purified by Flash silica gel column (A: DCM, B: MeOH) to obtain compound 1-5 (22.23 g, 73.00 mmol, yield: 77.16%). MS: 304.93 [M+H]⁺

### Step 5: Synthesis of Compound 1-6

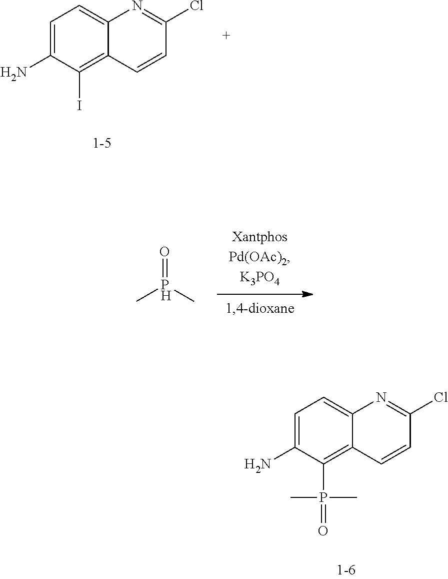

Compound 1-5 (10.00 g, 32.84 mmol), dimethylphosphine oxide (2.69 g, 34.48 mmol), Xantphos (3.80 g, 6.57 mmol), palladium acetate (737.27 mg, 3.28 mmol) and anhydrous potassium phosphate (13.94 g, 65.68 mmol) was dissolved in 1,4-dioxane (100 mL), replaced with nitrogen three times, heated to 100° C. and stirred overnight. Cool the temperature, filter the reaction solution, and concentrate the filtrate; the residue is purified on a Flash silica gel column, first with petroleum ether/ethyl acetate (ethyl acetate from 0-50%, run for 10 minutes), and then with dichloromethane/methanol (methanol 0-6%, 20 min) to obtain compound 1-6 (6.18 g, 24.27 mmol, yield: 73.90%). MS: 255.04 [M+H]⁺

### Step 6: Synthesis of Compound 1-7

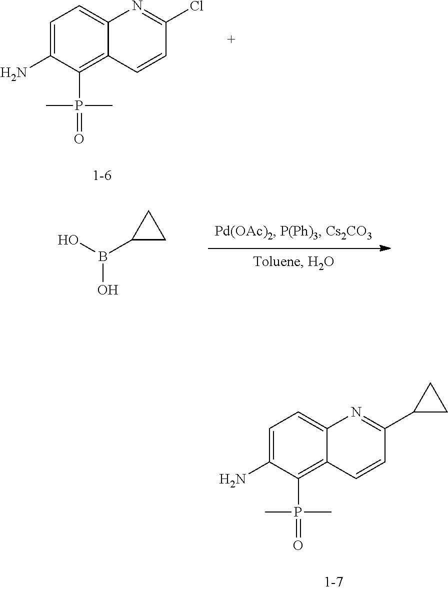 [SMILES: CP(C)(=O)c1c(N)ccc2nc(Cl)ccc12 ; OB(O)C1CC1 ; CP(C)(=O)c1c(N)ccc2nc(C3CC3)ccc12]

Compound 1-6 (4 g, 15.71 mmol), cyclopropylboronic acid (5.40 g, 62.83 mmol), palladium acetate (352.65 mg, 1.57 mmol), triphenylphosphine (0.82 g, 3.14 mmol), Cs₂CO₃ (15.35 g, 47.12 mmol), added to a mixed solvent of toluene (60 mL) and H₂O (10 mL), heated to 100° C. under nitrogen protection, and stirred for 12 h. The reaction was monitored for completion by LCMS and cooled to room temperature. Add 40 mL of water, separate the layers, take the organic phase, extract the aqueous phase with ethyl acetate (3×30 mL), combine the organic phases, dry over anhydrous sodium sulfate, filter, concentrate, and purify by column chromatography (dichloromethane: methanol)=15:1). Compound 1-7 (2.2 g, 8.45 mmol, yield: 53.81%) was obtained as a yellow solid. MS: 261.11 [M+H]⁺

### Step 7: Synthesis of Compound 1-8

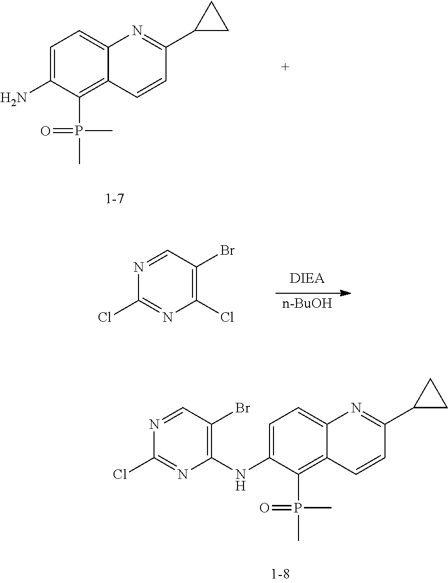

Add compound 1-7 (2.2 g, 8.45 mmol), 5-bromo-2,4-dichloropyrimidine (3.85 g, 16.91 mmol), DIEA (3.28 g, 25.36 mmol), n-BuOH (40 mL), heated to 120° C. and stirred for 10 h. LCMS monitors the reaction completion. The reaction was cooled to room temperature, filtered, and the filter cake was dried to obtain compound 1-8 (2.4 g, 5.31 mmol, yield: 62.86%), light yellow solid. MS: 451.00 [M+H]⁺

### Step 8: Synthesis of Compound 1-11

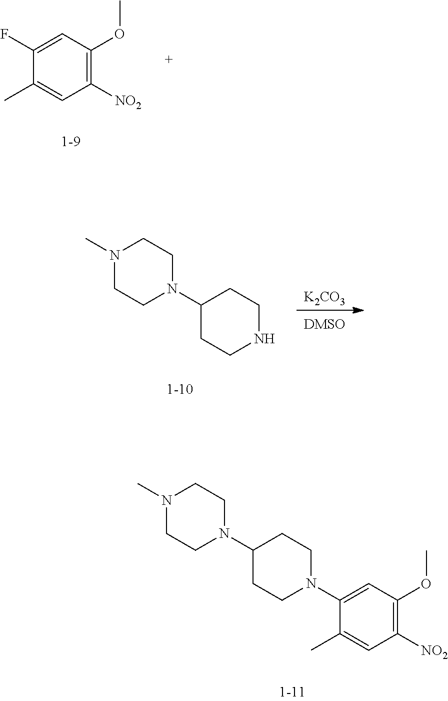

Add compound 1-9 (1 g, 5.40 mmol), compound 1-10 (1.19 g, 6.48 mmol), K₂CO₃ (1.49 g, 10.8 mmol) and DMSO (10 mL) to the reaction flask in sequence, and heat to 90° C. Heat and stir overnight. LCMS monitors the end of the reaction and stops the reaction. Add 50 mL DCM to the reaction solution, wash with water (100×2 mL), wash with 100 mL saturated brine, dry over anhydrous magnesium sulfate, concentrate, beat with diethyl ether, suction filter, and dry to obtain 1-11 (1.61 g, 4.62 mmol, product Rate: 85.55%), yellow solid. MS: 349.22[M+H]⁺

### Step 9: Synthesis of Compound 1-12

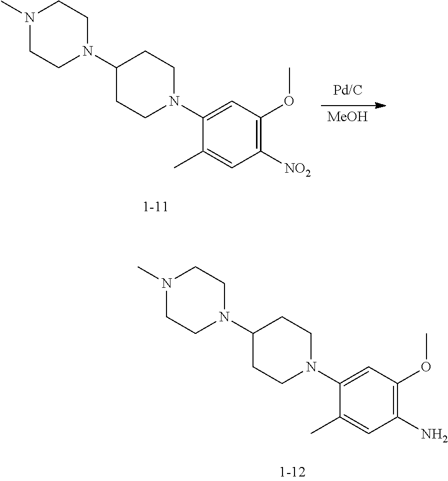

Compound 1-11 (1.61 g, 4.62 mmol), Pd/C (0.5 g, 10%) and MeOH (30 mL) were added to the reaction flask in sequence, H₂ was added, and the reaction solution was stirred at room temperature for 3 h. LCMS monitors the end of the reaction and stops the reaction. Filter with suction, rinse with methanol (20 mL), collect the organic phase, and remove the solvent to obtain the target product compound 1-12 (1.3 g, 4.08 mmol, yield: 88.31%). MS: 319.24[M+H]⁺

Step 10: Synthesis of the compound represented by formula I

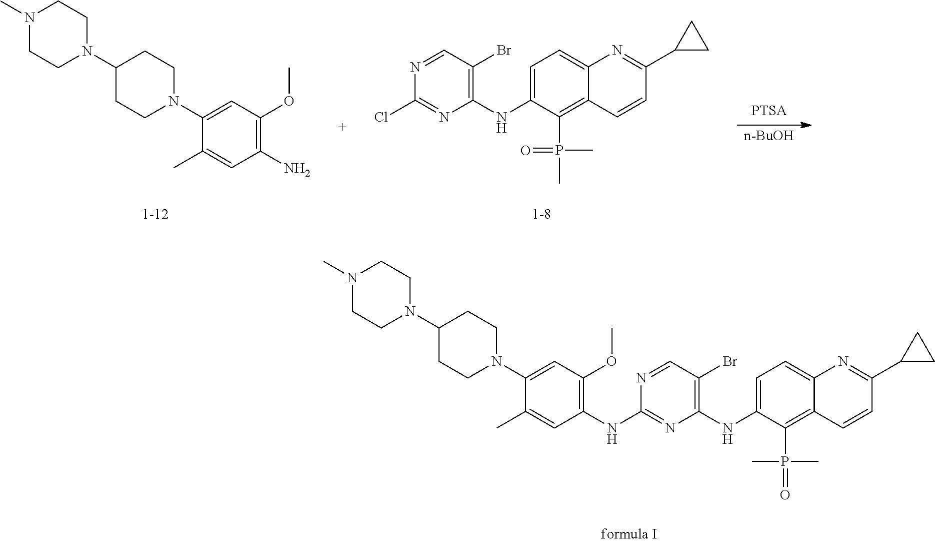

Add compound 1-12 (67.05 mg, 210.54 umol), compound 1-8 (104.61 mg, 231.59 umol), p-toluenesulfonic acid (65.26 mg, 378.97 umol) and n-BuOH (2 mL) in sequence to the reaction bottle. Heat to 100° C. and stir overnight. LCMS monitors that the reaction is complete, cool to room temperature, spin to dryness, add saturated Na₂CO₃ aqueous solution (10 mL), extract with dichloromethane (3×10 mL), dry over anhydrous sodium sulfate, filter and spin to dryness. The crude product was purified by PTLC (dichloromethane: methanol=10:1) to obtain the compound of formula I (24 mg, 32.71 umol, yield: 15.54%), which is an amorphous substance of formula I. MS: 733.27 [M+H]⁺

¹H-NMR (500 MHz, DMSO-d₆): δ 11.88 (s, 1H), 8.44 (d, J=8.5 Hz, 1H), 8.27-8.26 (m, 1H), 8.19 (s, 1H), 8.03 (s, 1H), 7.75 (d, J=9.0 Hz, 1H), 7.42 (d, J=8.5 Hz, 1H), 7.24 (br, 1H), 6.69 (s, 1H), 3.75 (s, 3H), 3.02-3.00 (m, 2H), 2.63-2.59 (m, 2H), 2.51-2.50 (m, 4H), 2.30-2.26 (m, 6H), 2.14 (s, 3H), 1.98 (d, J=13.5 Hz, 6H), 1.90 (s, 3H), 1.84-1.82 (m, 2H) 1.55-1.52 (m, 2H), 1.06-1.05 (m, 4H).

According to the above preparation method, the feeding amount is increased to obtain 20 g of the amorphous substance of the compound represented by formula I.

### Example 2: Synthesis of Crystal Form γ of the Compound Represented by Formula I

Add n-butanol (6 L), compound 1-8 (600.00 g) and compound 1-12 (634.50 g) to the 10 L reaction kettle. Stir and heat to 65±5° C. Add PTSA (573.51 g) to the reaction system, raise the temperature to 110±5° C. after addition, and keep the reaction warm. HPLC monitors until the reaction is complete, stops the reaction, and cools down. The reaction system was concentrated, and 9 L of dichloromethane was added to the concentrated residue to dissolve. The resulting solution was washed with hydrochloric acid aqueous solution (0.5 N) (6 L×2). The aqueous phases were combined and extracted with dichloromethane (6 L×2). The organic phase was Discard, and add 5 N sodium hydroxide aqueous solution to pH>10 while controlling the temperature of the aqueous phase below 25° C. with stirring. Then add dichloromethane for extraction (6 L×2). Combine the organic phases and add purified water for washing (6 L×2). The organic phase was concentrated under reduced pressure until about 3 L of solvent remained, then 3 L×2 acetonitrile was added, and the concentration was continued until about 3 L of solvent remained. Filter, and the filter cake is rinsed with acetonitrile (1.2 L×5) to obtain 849.11 g of crude product as a gray solid.

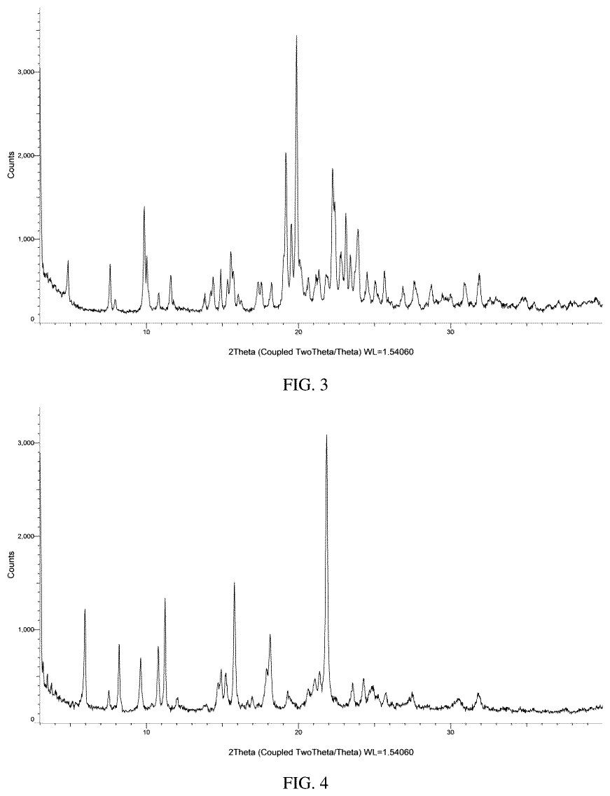

Dissolve 849.11 g of crude product in 6 L of methylene chloride and wash with L-malic acid aqueous solution (5.4 L×3, 0.2% Wt). After combining the aqueous phases, extract with dichloromethane (3 L×3), combine the organic phases, and wash the organic phase with potassium carbonate aqueous solution (6 L, 2% Wt) and purified water (6 L×2). Add 240.21 g of activated carbon and 360.71 g of palladium remover (SMA-90 5 metal remover) to the organic phase, stir under reflux for 2 hours, lower to room temperature, use diatomaceous earth to assist filtration, and rinse the filter cake with dichloromethane (3 L×4). The filtrate was concentrated under reduced pressure to about 3 L of solvent, then 3 L of ethyl acetate was added, and the concentration was continued until about 3 L of solvent remained. Repeat this operation once. The concentrated liquid was filtered, and the filter cake was rinsed with 3 L of ethyl acetate, and dried under reduced pressure at 25±5° C. to a constant weight to obtain 657.73 g of an off-white solid, which was identified by X-ray powder diffraction, showing that it was a compound of formula I. Crystal form γ, its XRPD spectrum is detailed in FIG. 3, and XRPD representative characteristic diffraction peak data is shown in Table 1.

TABLE 1
XRPD diffraction peaks of crystal form γ
of the compound represented by formula I

| | diffraction | Relative |
|---|---|---|
| No. | angle 2θ(°) | Strength(%) |
| 1 | 4.8 | 11.5 |
| 2 | 7.6 | 16.9 |
| 3 | 9.8 | 36.8 |
| 4 | 10.0 | 19.2 |
| 5 | 11.6 | 12.2 |
| 6 | 14.3 | 12.5 |
| 7 | 14.8 | 15.7 |
| 8 | 15.5 | 20.6 |
| 9 | 19.1 | 56.9 |
| 10 | 19.5 | 30.2 |
| 11 | 19.8 | 100.0 |
| 12 | 20.0 | 15.7 |
| 13 | 22.2 | 50.1 |
| 14 | 23.1 | 33.3 |
| 15 | 23.9 | 25.6 |

### Example 3: Synthesis of Crystal Form α, β, and δ of the Compound Represented by Formula I

Crystal form α: Add compound 1-12 (7.45 g, 23.38 mmol), compound 1-8 (8.80 g, 19.48 mmol), p-toluenesulfonic acid (8.39 g, 48.71 mmol) and n-BuOH (200 mL), heated to 100° C. and stirred overnight. Monitor the reaction is complete by LCMS, cool to room temperature, spin to dryness, add saturated Na₂CO₃ aqueous solution (10 mL), extract with dichloromethane (3×100 mL), dry with anhydrous sodium sulfate, filter and concentrate to 20 mL, add 50 mL acetonitrile to precipitate crystals. Filter, dry the crude product, separate through DCM:MeOH (8:1) chromatography column, concentrate to dryness, dissolve the solid in 30 mL of methylene chloride, then add 50 mL of acetonitrile, concentrate until a solid is about to appear, then add 10 mL EA was sonicated and filtered to obtain the compound of formula I (8.4 g, 11.47 mmol, yield: 58.88%). It was identified through X-ray powder diffraction, which showed that it was the crystal form α of the compound represented by Formula I. The details of its XRPD spectrum are shown in FIG. 1.

Crystal form β: Weigh 19.89 mg of the amorphous compound of formula I, place it in an HPLC vial, add 0.5 mL of acetone, and stir at room temperature for two days. The solid sample was centrifuged and dried under vacuum at 40° C. for 3 hours to obtain the compound of formula I. The compound was identified by X-ray powder diffraction, which showed that it was the crystal form β of the compound of formula I. Its XRPD spectrum is shown in FIG. 3. XRPD The representative characteristic diffraction peak data are shown in Table 2.

TABLE 2
XRPD diffraction peaks of crystal form β
of the compound represented by formula I

| | diffraction | Relative |
|---|---|---|
| No. | angle 2θ(°) | Strength(%) |
| 1 | 4.7 | 11.9 |
| 2 | 10.3 | 11.8 |
| 3 | 11.2 | 37.2 |
| 4 | 11.6 | 16.3 |
| 5 | 13.1 | 62.0 |
| 6 | 13.3 | 16.4 |
| 7 | 14.5 | 42.8 |
| 8 | 17.5 | 32.6 |
| 9 | 18.6 | 45.0 |
| 10 | 18.9 | 44.8 |
| 11 | 19.7 | 30.4 |
| 12 | 20.3 | 56.7 |
| 13 | 21.4 | 41.9 |
| 14 | 21.8 | 100.0 |

Crystal form δ: Weigh 19.99 mg of the amorphous compound of formula I and place it in an HPLC vial. Add 0.5 mL acetonitrile and stir at room temperature for two days. The solid sample was centrifuged and dried under vacuum at 40° C. for 3 hours to obtain the compound of formula I. It was identified through X-ray powder diffraction, showing that it is the crystal form δ of the compound represented by Formula I. Its XRPD spectrum is detailed in FIG. 4.

### Example 4: Synthesis of Crystal Form A of the Compound Represented by Formula III

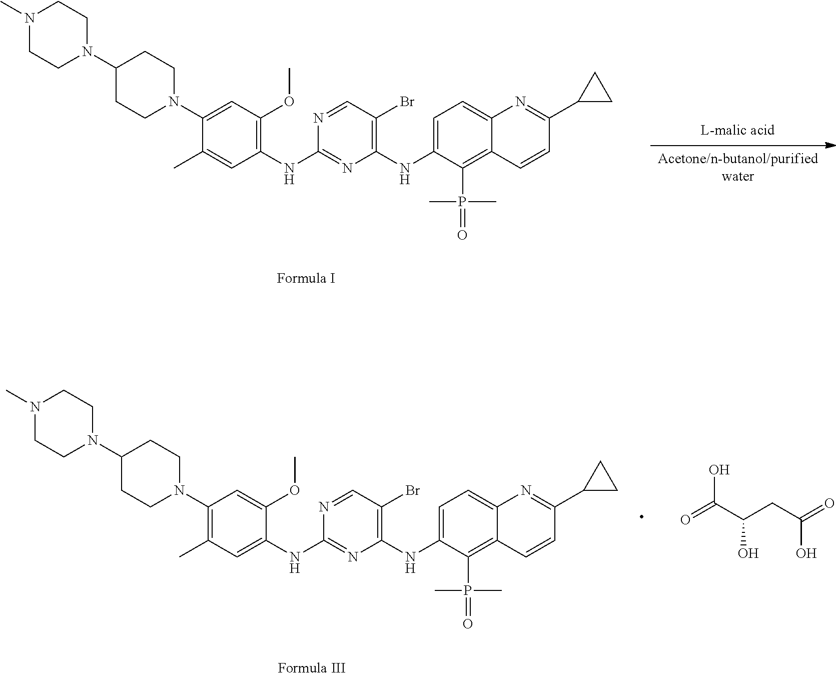 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1 ; COc1cc(N2CCC(N3CCN(C)CC3)CC2)c(C)cc1Nc1ncc(Br)c(Nc2ccc3nc(C4CC4)ccc3c2P(C)(C)=O)n1 ; O=C(O)C[C@H](O)C(=O)O]

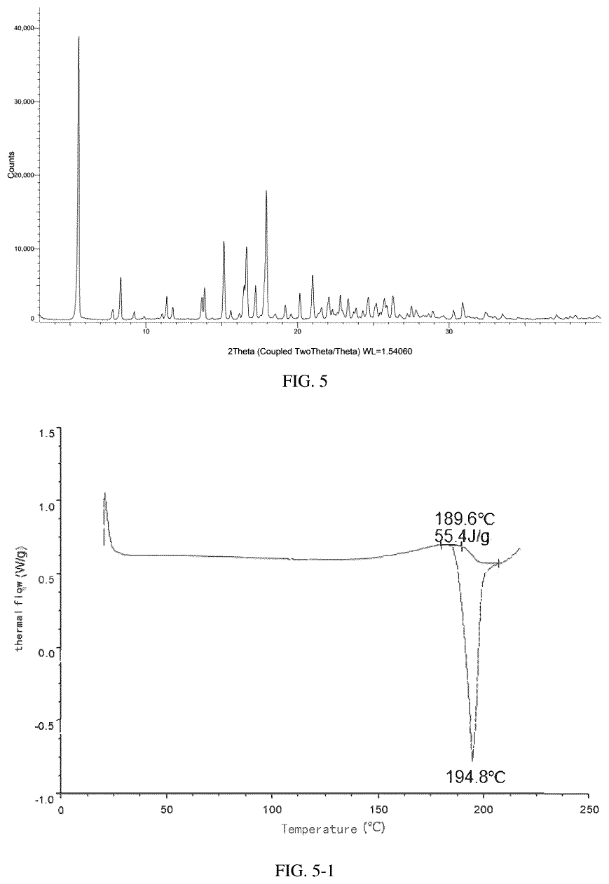

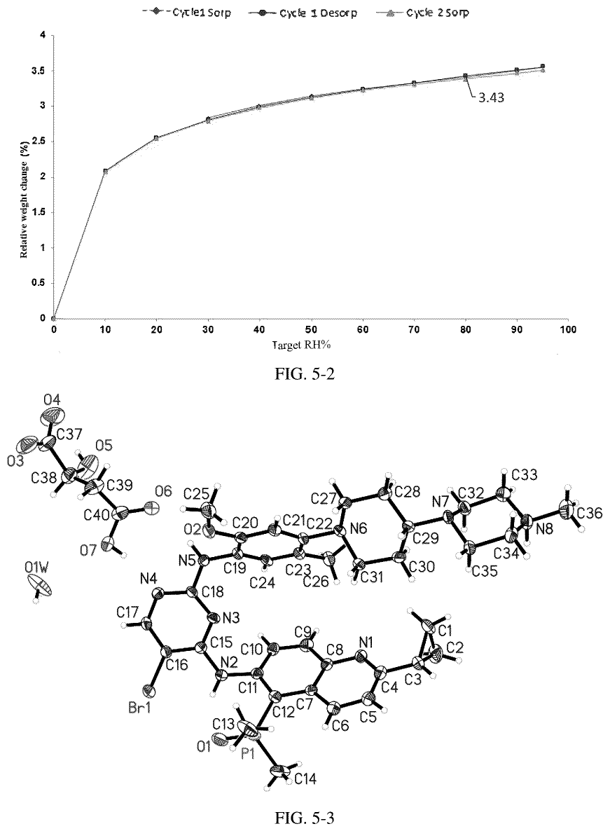

Method 1: Add acetone (21.7 L), n-butanol (3.10 L), purified water (0.62 L) and crystal form γ (619.13 g) of the compound shown in formula I into a 50 L reaction kettle, stir and heat to reflux. The acetone solution of L-malic acid was added dropwise to the reaction system (59.40 g L-malic acid was dissolved in 0.62 L acetone). After the dropwise addition, the acetone solution of L-malic acid was added dropwise (59.41 g L-malic acid was dissolved in 0.62 L acetone). 0.62 L acetone). After the dropwise addition is completed, keep the temperature for 0.5 h, cool to 30-35° C. and filter. The filter cake is rinsed with 6.2 L acetone, vacuum dried at 25±5° C., weighed to obtain 621.47 g of off-white solid, and identified by X-ray powder diffraction. It was shown that it is the crystal form A of the compound represented by formula III, and its moisture content was measured to be 3.51%; DSC was also measured, and the obtained DSC spectrum is shown in FIG. 5-1.

The XRPD spectrum is detailed in FIG. 5, and the XRPD representative characteristic diffraction peak data is shown in Table 3.

TABLE 3
XRPD diffraction peaks of crystal form A of the compound
represented by formula III

| | diffraction angle | Relative |
|---|---|---|
| No. | 2θ(°) | Strength (%) |
| 1 | 5.5 | 100.0 |
| 2 | 7.8 | 3.8 |
| 3 | 8.3 | 15.9 |
| 4 | 9.2 | 2.9 |
| 5 | 11.3 | 8.9 |
| 6 | 11.7 | 4.5 |
| 7 | 13.6 | 8.4 |
| 8 | 13.8 | 12.5 |
| 9 | 15.1 | 30.5 |
| 10 | 16.4 | 12.9 |
| 11 | 16.6 | 28.0 |
| 12 | 17.2 | 13.3 |
| 13 | 17.9 | 51.3 |
| 14 | 20.1 | 10.7 |
| 15 | 20.9 | 17.9 |

Its NMR data are as follows: ¹H-NMR (500 MHz, DMSO-d₆): δ11.84 (s, 1H), 8.47 (d, 1H), 8.27-8.26 (m, 1H), 8.19 (s, 1H), 8.01 (s, 1H), 7.76 (d, 1H), 7.43 (d, 1H), 7.26 (s, 1H), 6.70 (s, 1H), 4.03 (dd, 1H), 3.75 (s, 3H), 3.05-3.03 (m, 2H), 2.74 (br, 8H), 2.65-2.61 (m, 2H), 2.45 (s, 3H), 2.47-2.45 (m, 1H), 2.46 (dd, 2H), 2.31-2.26 (m, 1H), 1.98 (d, 6H), 1.90 (s, 3H), 1.90-1.87 (m, 2H), 1.62-1.55 (m, 2H), 1.07-1.06 (m, 4H).

Method 2: Weigh 19.70 mg of the amorphous compound of formula I and 3.86 mg of L-malic acid and place them in an HPLC vial. Add 0.5 mL of tetrahydrofuran/water (19:1, v/v) mixed solvent and stir at room temperature for two days. The solid sample was centrifuged and dried under vacuum at 40° C. for 3 hours to obtain crystal form A of the compound represented by formula III, which has the same XRPD pattern as the method one of Example 4.

Method 3: Weigh 5 mg of crystal form A of the compound shown in formula III, add it to 1 mL of isopropyl alcohol, stir at room temperature to dissolve, then filter. Transfer the filtrate to a glass vial, cover it with a plastic film, poke a small hole, and place at room temperature. Under slow volatilization, a single crystal sample of Form A of the compound represented by Formula III is obtained, which has the same XRPD pattern as Method 1 of Example 4.

Method 4: Add acetone (26 L), purified water (0.65 L) and crystal form γ (650.00 g) of the compound shown in Formula I to the 50 L reaction kettle, stir and raise the temperature to 55-60° C. The acetone solution of L-malic acid was added dropwise to the reaction system (124.70 g L-malic acid was dissolved in 1.3 L acetone), and after the dropwise addition was completed, the crystal form A (28.40 g) of the compound represented by the seed crystal formula III was added. Stir for 0.5 h, cool to room temperature and filter. The filter cake is rinsed with 6 L of acetone, dried under vacuum at 65±5° C., and weighed to obtain 685.00 g of off-white solid. That is, the crystal form A of the compound represented by Formula III is obtained, which has the same characteristics as the XRPD pattern in Examples 4 method 1. Its moisture content was measured to be 1.08%.

Method 5: Add acetone (36.75 L), n-butanol (5.25 L), purified water (1.05 L) and crystal form γ of the compound represented by formula I (1050.08 g) into a 100 L reaction kettle, stir and heat to reflux. Add the acetone solution of L-malic acid dropwise to the reaction system (100.74 g L-malic acid is dissolved in 1.05 L acetone). After the dropwise addition is completed, add crystal form A (21.00 g) of the compound shown in the seed formula III and continue to dropwise Acetone solution of L-malic acid (100.75 g L-malic acid dissolved in 1.05 L acetone). After the dropwise addition is completed, keep the temperature for 0.5 h, cool to 30-35° C. and filter. The filter cake is rinsed with 10.5 L acetone, vacuum dried at 25±5° C., and weighed to obtain 1040.71 g of off-white solid, which is the crystal form of the compound shown in formula III. A, which has the same XRPD pattern as Method 1 of Example 4. Its moisture content was measured to be 3.45%.

Method 6: Add acetone (37.60 kg), n-butanol (5.50 kg), purified water (1.36 kg) and crystal form γ of the compound represented by formula I (1.36 kg) into a 100 L reaction kettle, stir and raise the temperature to reflux. The acetone solution of L-malic acid was added dropwise to the reaction system (0.13 kg L-malic acid was dissolved in 1.07 kg acetone), and after the dropwise addition was completed, the crystal form A (0.03 kg) of the compound represented by the seed crystal formula III was added. Continue to add the L-malic acid acetone solution dropwise (0.13 kg L-malic acid dissolved in 1.08 kg acetone). After the dropwise addition is completed, keep it warm for 0.5 h, cool to 30-35° C. and filter. The filter cake is rinsed with 10.75 kg acetone, dried under vacuum at 25±5° C., and weighed to obtain 1.36 kg of off-white solid, thus obtaining the crystal form A of the compound shown in formula III, which has the same XRPD pattern as Method 1 of Example 4. Its moisture content was measured to be 3.70%.

### Example 5: Synthesis of Form B of the Compound of Formula III

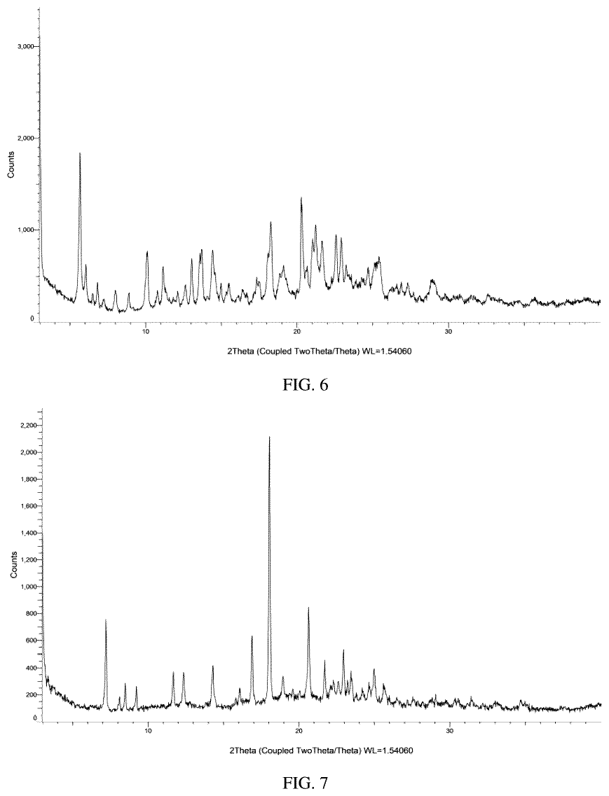

Weigh 20.01 mg of the amorphous compound of formula I and 3.96 mg of L-malic acid, and place them in an HPLC vial. Add 0.5 mL acetonitrile and stir at room temperature for two days. The solid sample was centrifuged and dried under vacuum at 40° C. for 3 hours to obtain the compound of formula III. It was identified through X-ray powder diffraction, showing that it is the crystal form B of the compound represented by formula III. Its XRPD spectrum is shown in FIG. 6, and the XRPD representative characteristic diffraction peak data is shown in Table 4.

TABLE 4
XRPD diffraction peaks of crystalline form B of the
compound represented by formula III

| | diffraction angle | Relative |
|---|---|---|
| No. | 2θ(°) | Strength (%) |
| 1 | 5.6 | 100.0 |
| 2 | 6.0 | 26.3 |
| 3 | 10.0 | 37.5 |
| 4 | 11.1 | 27.3 |
| 5 | 13.0 | 32.8 |
| 6 | 13.5 | 35.9 |
| 7 | 13.7 | 36.6 |
| 8 | 14.4 | 36.6 |
| 9 | 18.0 | 33.1 |
| 10 | 18.2 | 57.1 |
| 11 | 19.0 | 24.0 |
| 12 | 20.2 | 70.6 |
| 13 | 20.6 | 23.2 |
| 14 | 21.0 | 39.0 |
| 15 | 21.2 | 51.4 |
| 16 | 21.6 | 38.1 |
| 17 | 22.5 | 42.9 |
| 18 | 22.9 | 42.1 |
| 19 | 23.2 | 22.6 |
| 20 | 24.6 | 21.4 |
| 21 | 25.4 | 29.3 |

### Example 6: Synthesis of Form C of the Compound of Formula III

Weigh 2.00 g of the crystal form α of the compound represented by Formula I and 366.35 mg of L-malic acid into a 100 mL single-mouth bottle containing 40 mL of acetone, stir at room temperature for 7 hours, then filter, and dry the wet product under vacuum at 50° C. for 11 hours. Then vacuum dry at 60° C. for 5 hours to obtain the compound represented by formula III. Identification by X-ray powder diffraction showed that it is the crystal form C of the compound represented by Formula III. Its XRPD spectrum is shown in FIG. 7. The XRPD representative characteristic diffraction peak data is shown in Table 5.

TABLE 5
XRPD diffraction peaks of crystal form C of the
compound represented by formula III

| | diffraction angle | Relative |
|---|---|---|
| No. | 2θ(°) | Strength (%) |
| 1 | 7.2° | 31.6% |
| 2 | 8.1° | 4.9% |
| 3 | 8.4° | 9.3% |
| 4 | 9.2° | 8.3% |
| 5 | 11.6° | 12.0% |
| 6 | 12.3° | 12.4% |
| 7 | 14.2° | 15.2% |
| 8 | 16.8° | 23.8% |
| 9 | 18.0° | 100.0% |
| 10 | 20.6° | 35.2% |

### Example 7: Synthesis of Crystal Form D of the Compound Represented by Formula III

Weigh 80 mg of crystal form α of the compound represented by Formula I and dissolve it in 2 mL of anhydrous methanol, heat to 60° C., and stir to dissolve. Slowly add 1 mL of methanol solution of L-malic acid dropwise (weigh 147.32 mg of L-malic acid and dissolve it in 10 mL of methanol), cool down to precipitate the solid, stir at room temperature for 5 hours and then centrifuge, and dry the wet product under vacuum at 50° C. for 11 hours to obtain compounds of formula III. It was identified through X-ray powder diffraction, which showed that it is the crystal form D of the compound represented by Formula III. Its XRPD spectrum is shown in FIG. 8. The XRPD representative characteristic diffraction peak data is shown in Table 5.

TABLE 5
XRPD diffraction peaks of crystalline form D of the
compound represented by formula III

| | diffraction angle | Relative |
|---|---|---|
| No. | 2θ(°) | Strength (%) |
| 1 | 5.4 | 100.0 |
| 2 | 8.3 | 17.7 |
| 3 | 14.8 | 47.6 |
| 4 | 16.4 | 34.4 |
| 5 | 16.6 | 34.1 |
| 6 | 17.6 | 91.9 |
| 7 | 25.2 | 26.2 |

### Example 8: Synthesis of Crystal Form E of the Compound Represented by Formula III

Weigh 80 mg of the crystal form α of the compound represented by Formula I and dissolve it in 2 mL of absolute ethanol, heat to 60° C., and stir to dissolve. Slowly add 1 mL of L-malic acid ethanol solution dropwise (weigh 146.46 mg L-malic acid dissolved in 10 mL of ethanol), cool down to precipitate the solid, stir at room temperature for 5 hours and then centrifuge, and dry the wet product under vacuum at 50° C. for 11 hours to obtain crystal form E of the compound represented by formula III. It was identified through X-ray powder diffraction, showing that it is the crystal form E of the compound represented by Formula III. Its XRPD spectrum is shown in FIG. 9, and the XRPD representative characteristic diffraction peak data is shown in Table 6.

TABLE 6
XRPD diffraction peaks of crystalline form E of the
compound represented by formula III

| | diffraction angle | Relative |
|---|---|---|
| No. | 2θ(°) | Strength (%) |
| 1 | 7.1° | 27.4% |
| 2 | 11.9° | 10.8% |
| 3 | 14.3° | 20.1% |
| 4 | 15.1° | 12.3% |
| 5 | 15.9º | 11.2% |
| 6 | 19.3° | 12.1% |
| 7 | 20.5° | 100.0% |
| 8 | 26.7º | 10.3% |

### Example 9: Synthesis of Form F of the Compound of Formula III

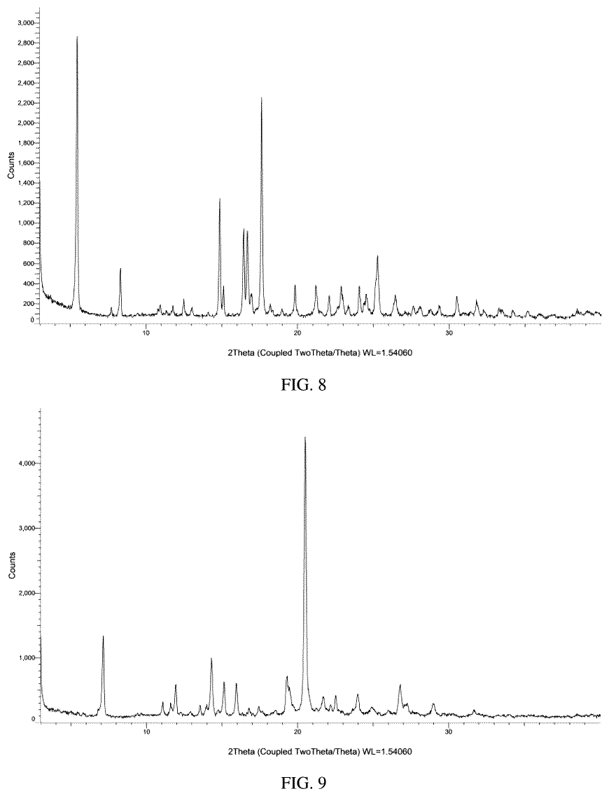

Weigh 5.03 g of crystal form α of the compound represented by Formula I and dissolve it in 75 mL of tetrahydrofuran, heat to reflux, and stir to dissolve. Weigh 1.03 g L-malic acid and dissolve it in 25 mL tetrahydrofuran, quickly add it to the reaction solution, react under reflux for 4 hours, cool to room temperature and then filter with suction to obtain the compound of formula III. It was identified through X-ray powder diffraction, showing that it is the crystal form F of the compound represented by Formula III. Its XRPD spectrum is shown in FIG. 10, and the XRPD representative characteristic diffraction peak data is shown in Table 7.

TABLE 7
XRPD diffraction peaks of crystalline form F of the
compound represented by formula III

| | diffraction angle | Relative |
|---|---|---|
| No. | 2θ(°) | Strength (%) |
| 1 | 6.6 | 43.6 |
| 2 | 7.4 | 24.2 |
| 3 | 10.5 | 25.8 |
| 4 | 16.4 | 33.8 |
| 5 | 21.1 | 100.0 |
| 6 | 22.1 | 28.7 |
| 7 | 22.7 | 32.1 |
| 8 | 24.4 | 23.1 |
| 9 | 26.9 | 19.8 |

### Example 10: Synthesis of Crystalline Form G of the

Compound Represented by Formula III Method 1: Weigh 1.00 g of the crystal form γ of the compound shown in Formula I and add it to a 100 mL single-mouth bottle containing 23 mL of acetone. Add L-malic acid solution (0.18 g dissolved in 2 mL of acetone), stir at room temperature for 2 days, and then pump. Filter and dry to obtain the compound of formula III. It was identified through X-ray powder diffraction, which showed that it is the crystal form G of the compound represented by Formula III. Its XRPD spectrum is shown in FIG. 11, and the XRPD representative characteristic diffraction peak data is shown in Table 8.

TABLE 8
XRPD diffraction peaks of crystalline form G of the
compound represented by formula III

| | diffraction angle | Relative |
|---|---|---|
| No. | 2θ(°) | Strength (%) |
| 1 | 5.0 | 85.7 |
| 2 | 10.0 | 100.0 |
| 3 | 15.0 | 83.1 |
| 4 | 19.5 | 30.5 |

Method 2: Weigh about 20 mg L-malate crystal form A sample, place it in an HPLC vial, add 0.5 mL acetone or isopropyl alcohol, suspend and stir at 50° C. for 4 days, separate the solid, and vacuum dry at 50° C. for 12 hours. Afterwards, the compound represented by formula III is obtained and identified by X-ray powder diffraction. Its XRPD pattern has the same or similar characteristic peaks as the XRPD pattern of the crystal form G obtained by method 1, so it is also the crystal form G of the compound represented by formula III.

### Example 11: Synthesis of Crystal Form H of the Compound Represented by Formula III

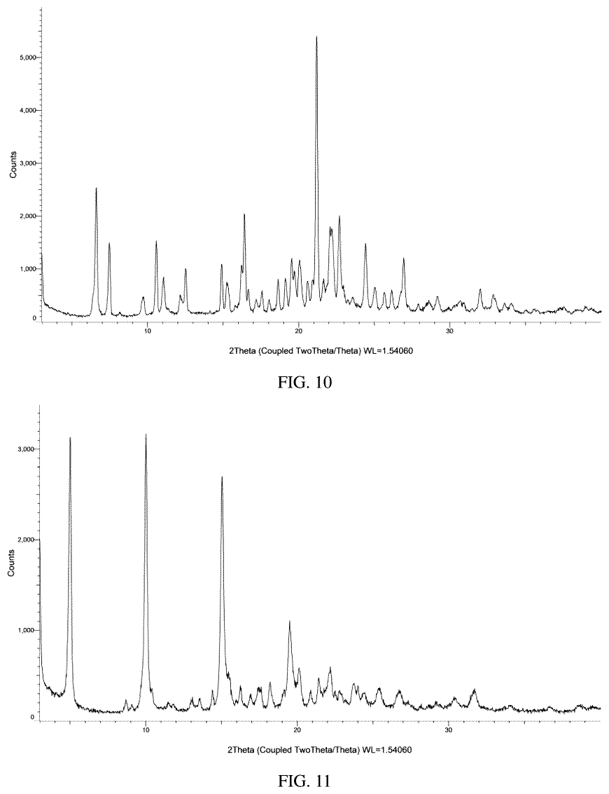

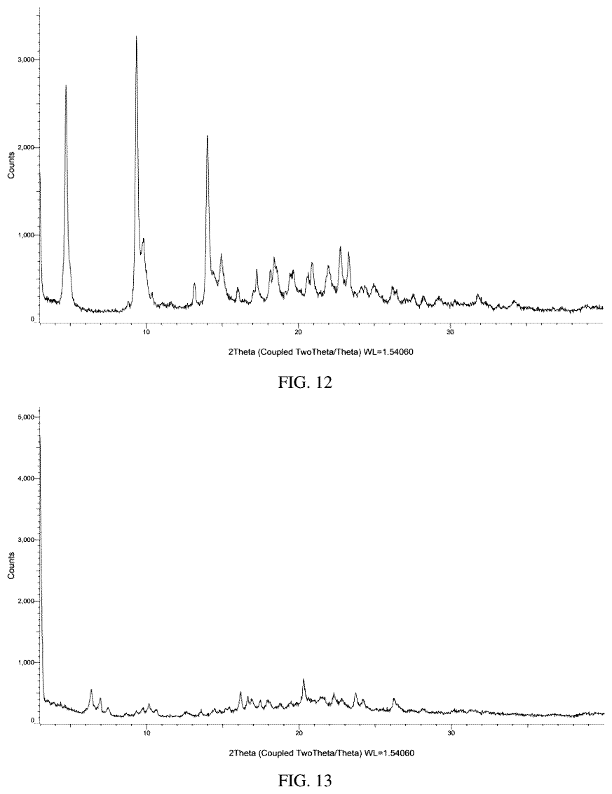

Method 1: Weigh 2.5 g of the crystal form γ of the compound represented by formula I into a 100 mL single-mouth bottle, add 50 mL of absolute ethanol and stir to dissolve, then weigh 482 mg of L-malic acid and dissolve it in 10 mL of ethanol, and add slowly dropwise into the reaction solution, and the solid was precipitated through seeding induction, stirred overnight and then filtered with suction, and the wet product was vacuum dried at 50° C. for 5 hours to obtain the compound represented by formula III. It was identified through X-ray powder diffraction, showing that it is the crystal form H of the compound represented by formula III. Its XRPD spectrum is shown in FIG. 12. The representative data of XRPD spectrum analysis is shown in Table 9.

TABLE 9
XRPD diffraction peaks of crystal form H of the
compound represented by Formula III

| | diffraction angle | Relative |
|---|---|---|
| No. | 2θ(°) | Strength (%) |
| 1 | 4.7 | 71.8 |
| 2 | 9.3 | 100.0 |
| 3 | 9.7 | 23.5 |
| 4 | 14.0 | 61.2 |
| 5 | 14.9 | 18.3 |
| 6 | 17.2 | 12.0 |
| 7 | 18.4 | 12.0 |
| 8 | 22.7 | 17.6 |
| 9 | 23.3 | 17.1 |

Method 2: Weigh about 20 mg L-malate crystal form A sample, place it in an HPLC vial, add 0.5 mL ethanol, suspend and stir at room temperature for 4 days, separate the solid, and vacuum dry at 50° C. for 12 hours to obtain formula III. The compound shown was identified by X-ray powder diffraction. Its XRPD pattern has the same or similar characteristic peaks as the XRPD pattern of the crystalline form H obtained by method 1, so it is also the crystalline form H of the compound represented by formula III.

### Example 12: Synthesis of Crystal Form I of the Compound Represented by Formula III

Weigh 80.36 mg of the crystal form γ of the compound represented by Formula I into an HPLC vial, add 2 mL of acetonitrile and 15.14 mg of L-malic acid, and stir at room temperature overnight. The solid was separated by centrifugation, and the wet product was vacuum dried at 50° C. for 12 hours to obtain the compound of formula III. It was identified through X-ray powder diffraction, which showed that it was Form I of the compound represented by Formula III. Its XRPD spectrum is detailed in FIG. 13.

### Example 13: Synthesis of Crystal Form J of the Compound Represented by Formula III

Weigh 2.00 g of crystal form A of the compound represented by Formula III into a 100 mL jacketed reaction kettle, and add 50 mL of acetone. Raise the temperature to 65° C. and stir for 2 hours. Slowly lower to room temperature and then filter with suction. The wet product is vacuum dried at 60° C. for 2.5 hours to obtain the compound represented by Formula III. It was identified through X-ray powder diffraction, which showed that it is the crystal form J of the compound represented by formula III. Its XRPD spectrum is detailed in FIG. 14, and the XRPD representative characteristic diffraction peak data is shown in Table 10.

TABLE 10
XRPD diffraction peaks of crystal form J of the
compound represented by formula III

| | diffraction angle | Relative |
|---|---|---|
| No. | 2θ(°) | Strength (%) |
| 1 | 9.0 | 77.7 |
| 2 | 11.2 | 43.6 |
| 3 | 11.7 | 32.8 |
| 4 | 12.2 | 30.6 |
| 5 | 14.0 | 31.7 |
| 6 | 15.5 | 51.1 |
| 7 | 16.2 | 20.5 |
| 8 | 18.0 | 41.4 |
| 9 | 19.2 | 20.1 |
| 10 | 20.0 | 63.0 |
| 11 | 20.4 | 49.7 |
| 12 | 21.3 | 100.0 |
| 13 | 23.1 | 26.0 |
| 14 | 24.6 | 42.7 |
| 15 | 25.5 | 38.9 |

### Example 14: Synthesis of Form A of the Compound Formula IV

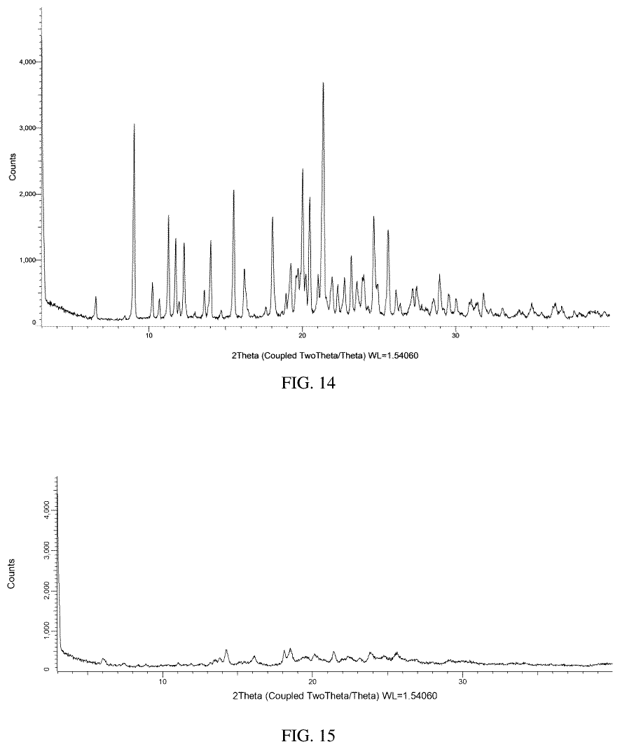

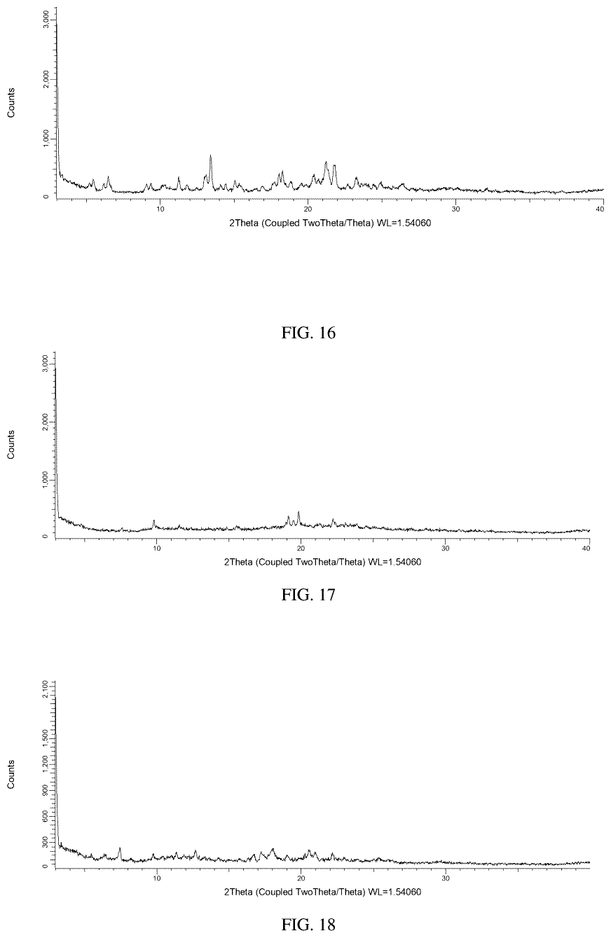

Weigh approximately 200 mg of the crystal form γ of the compound represented by Formula I into a 10 mL vial, add 4 mL of ethanol and 110.23 mg of L-malic acid, and stir at room temperature for three days. The solid was separated by centrifugation, and the wet product was vacuum dried at 50° C. for 12 hours to obtain the compound of formula IV. It was identified through X-ray powder diffraction, showing that it is the crystal form A of the compound represented by Formula IV. Its XRPD spectrum is shown in FIG. 16, and the XRPD representative characteristic diffraction peak data is shown in Table 11.

TABLE 11
XRPD diffraction peaks of crystalline form A of the
compound represented by formula IV

| | diffraction angle | Relative |
|---|---|---|
| No. | 2θ(°) | Strength (%) |
| 1 | 5.5 | 23.7 |
| 2 | 6.2 | 19.5 |
| 3 | 6.5 | 39.3 |
| 4 | 9.1 | 21.4 |
| 5 | 9.4 | 24.7 |
| 6 | 11.2 | 41.7 |
| 7 | 13.1 | 43.1 |
| 8 | 13.4 | 100.0 |
| 9 | 15.1 | 29.0 |
| 10 | 18.0 | 45.8 |
| 11 | 18.2 | 59.1 |
| 12 | 19.5 | 19.3 |
| 13 | 20.4 | 41.2 |
| 14 | 21.2 | 75.7 |
| 15 | 21.3 | 51.1 |
| 16 | 21.7 | 69.6 |
| 17 | 23.3 | 30.0 |
| 18 | 24.9 | 25.6 |

### Example 15: Synthesis of Form B of the Compound Formula IV

Weigh approximately 200 mg of the crystal form γ of the compound represented by Formula I into a 10 mL vial, add 4 mL of isopropyl alcohol and 110.92 mg of L-malic acid, and stir at room temperature for three days. The solid was separated by centrifugation, and the wet product was vacuum dried at 50° C. for 12 hours to obtain the compound of formula IV. It was identified through X-ray powder diffraction, showing that it is the crystal form B of the compound represented by Formula IV. Its XRPD spectrum is shown in FIG. 17, and the XRPD representative characteristic diffraction peak data is shown in Table 12.

TABLE 12
XRPD diffraction peaks of crystalline form B of the
compound represented by formula IV

| | diffraction angle | Relative |
|---|---|---|
| No. | 2θ(°) | Strength (%) |
| 1 | 7.6 | 21.4 |
| 2 | 9.8 | 56.1 |
| 3 | 11.6 | 31.0 |
| 4 | 19.1 | 71.6 |
| 5 | 19.5 | 46.0 |
| 6 | 19.8 | 100.0 |
| 7 | 21.3 | 26.2 |
| 8 | 22.2 | 62.3 |
| 9 | 23.1 | 20.3 |

### Example 16: Synthesis of Form C of the Compound of Formula IV

Weigh about 50 mg of the crystal form γ of the compound represented by formula I into an HPLC vial, add 1 mL of tetrahydrofuran and 19.27 mg of L-malic acid, and stir at room temperature for three days. The solid was separated by centrifugation, and the wet product was vacuum dried at 50° C. for 12 hours to obtain the compound of formula IV. It was identified through X-ray powder diffraction, showing that it is the crystal form C of the compound represented by Formula IV. Its XRPD spectrum is shown in FIG. 18. The XRPD representative characteristic diffraction peak data is shown in Table 13.

TABLE 13
XRPD diffraction peaks of crystalline form C of the
compound represented by formula IV

| | diffraction angle | Relative |
|---|---|---|
| No. | 2θ(°) | Strength (%) |
| 1 | 8.0 | 29.6 |
| 2 | 8.7 | 30.0 |
| 3 | 12. | 100.0 |
| 4 | 21.9 | 55.3 |

### Example 17: Synthesis of Crystal Form A of the Compound Represented by Formula V

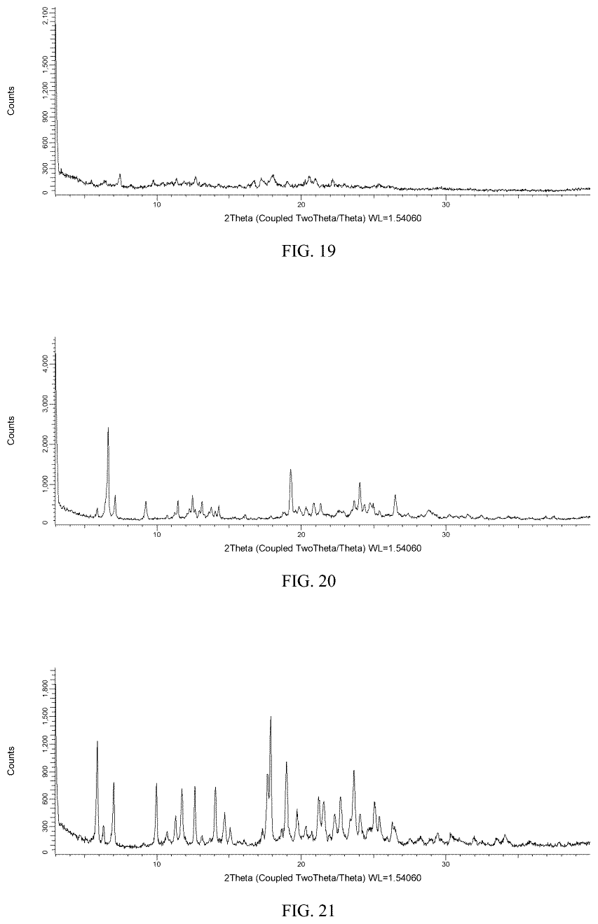

Weigh about 50 mg of the crystal form γ of the compound represented by formula I into an HPLC vial, add 1 mL acetone/water (19:1, v/v) and 19.27 mg L-malic acid, stir at room temperature for four days, and then raise the temperature to 50° C. for 20 hours, then returned to room temperature, and obtained a solid after two temperature cycles. The solid was separated by centrifugation, and the wet product was vacuum dried at 50° C. for 9 hours to obtain the compound of formula IV. It was identified through X-ray powder diffraction, showing that it is the crystal form A of the compound represented by formula V. Its XRPD spectrum is shown in FIG. 19, and the XRPD representative characteristic diffraction peak data is shown in Table 14.

TABLE 14
XRPD diffraction peaks of crystalline form A of the
compound represented by formula V

| | diffraction angle | Relative |
|---|---|---|
| No. | 2θ(°) | Strength (%) |
| 1 | 6.4 | 24.6 |
| 2 | 7.4 | 100.0 |
| 3 | 9.7 | 31.2 |
| 4 | 11.4 | 58.1 |
| 5 | 12.7 | 66.1 |
| 6 | 16.7 | 39.2 |
| 7 | 18.0 | 85.3 |
| 8 | 19.0 | 40.9 |
| 9 | 20.5 | 62.5 |
| 10 | 21.0 | 54.5 |
| 11 | 22.2 | 57.0 |
| 12 | 23.0 | 18.6 |

### Example 18: Synthesis of Hydrochloride Crystal Form B of the Compound Represented by Formula I

Weigh about 20 mg of the hydrochloride crystal form A of the compound represented by Formula I, add 0.5 mL of anhydrous methanol into an HPLC vial, and suspend and stir at room temperature for 1 day. The solid was centrifuged and dried under vacuum at 50° C. for 3 hours to obtain the monohydrochloride of the compound represented by Formula I. It was identified through X-ray powder diffraction, showing that it is the hydrochloride crystal form B of the compound represented by formula I. Its XRPD spectrum is shown in FIG. 20, and the XRPD representative characteristic diffraction peak data is shown in Table 15.

TABLE 15
XRPD diffraction peaks of monohydrochloride crystal
form B of the compound represented by formula I

| | diffraction angle | Relative |
|---|---|---|
| No. | 2θ(°) | Strength (%) |
| 1 | 6.6 | 100.0 |
| 2 | 7.1 | 25.7 |
| 3 | 9.2 | 19.2 |
| 4 | 11.4 | 19.8 |
| 5 | 12.5 | 27.6 |
| 6 | 13.1 | 18.9 |
| 7 | 19.3 | 56.8 |
| 8 | 23.7 | 18.9 |
| 9 | 24.0 | 40.8 |
| 10 | 26.5 | 27.3 |

### Example 19: Synthesis of L-Tartrate Crystal Form A of the Compound Represented by Formula III

Weigh about 800 mg of the crystal form α of the compound represented by formula I and 163.97 mg of L-tartaric acid, add 10 mL of acetonitrile solvent, stir magnetically at room temperature overnight, centrifuge to obtain a solid, and vacuum dry at 50° C. for 4 hours to obtain compound L-tartrate represented by formula I. It was identified through X-ray powder diffraction, showing that it is the compound L-tartrate crystal form A represented by formula I. Its XRPD spectrum is detailed in FIG. 21, and the XRPD representative characteristic diffraction peak data is shown in Table 16.

TABLE 16
XRPD diffraction peaks of compound L-tartrate crystal
form A represented by formula I

| | diffraction angle | Relative |
|---|---|---|
| No. | 2θ(°) | Strength (%) |
| 1 | 5.8 | 73.0 |
| 2 | 7.0 | 45.8 |
| 3 | 9.9 | 47.5 |
| 4 | 11.7 | 42.7 |
| 5 | 12.6 | 47.1 |
| 6 | 14.0 | 42.5 |
| 7 | 14.7 | 25.9 |
| 8 | 17.6 | 54.4 |
| 9 | 17.8 | 100.0 |
| 10 | 18.9 | 61.7 |
| 11 | 21.2 | 33.8 |
| 12 | 21.5 | 30.0 |
| 13 | 22.7 | 33.8 |
| 14 | 23.6 | 54.6% |

### Example 20: Synthesis of Fumarate Crystal Form B of the Compound Represented by Formula I

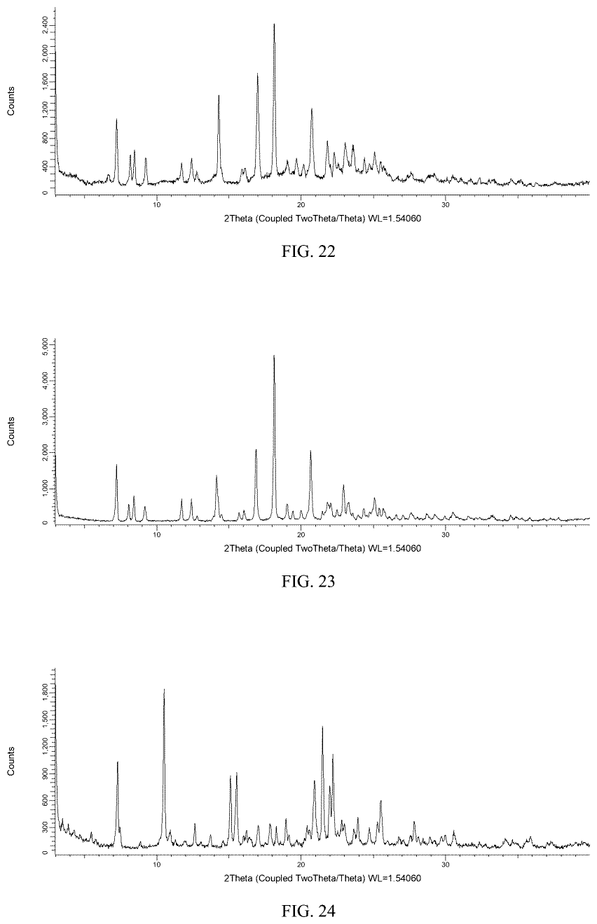

Weigh about 400 mg of the crystal form α of the compound represented by formula I and 63.42 mg of fumaric acid, add 8 mL of acetone solvent, stir magnetically at room temperature overnight, centrifuge to obtain a solid, and dry it under vacuum at 50° C. for 4 hours. The fumarate salt of the compound represented by formula I is obtained. It was identified through X-ray powder diffraction, showing that it is the fumarate crystal form B of the compound represented by Formula I. Its XRPD spectrum is shown in FIG. 22, and the XRPD representative characteristic diffraction peak data is shown in Table 17.

TABLE 17
XRPD diffraction peaks of fumarate crystal form B
of the compound represented by formula I

| | diffraction angle | Relative |
|---|---|---|
| No. | 2θ (°) | Strength (%) |
| 1 | 7.2 | 40.6 |
| 2 | 8.1 | 17.2 |
| 3 | 8.4 | 21.7 |
| 4 | 9.2 | 15.5 |
| 5 | 14.3 | 55.6 |
| 6 | 17.0 | 65.3 |
| 7 | 18.1 | 100.0 |
| 8 | 20.7 | 44.0 |

### Example 21: Synthesis of Succinate Crystal Form A of the Compound Represented by Formula I

Weigh about 400 mg of the crystal form α of the compound represented by formula I and 64.64 mg of succinic acid, add 10 mL of acetone solvent, stir magnetically at room temperature overnight, centrifuge to obtain a solid, and vacuum dry it at 50° C. for 9 hours to obtain compound I succinate. It was identified through X-ray powder diffraction, showing that it is the succinate crystal form A of the compound represented by Formula I. Its XRPD spectrum is shown in FIG. 23, and the XRPD representative characteristic diffraction peak data is shown in Table 18.

TABLE 18
XRPD diffraction peaks of succinate crystal form A
of the compound represented by formula I

| | diffraction angle | Relative |
|---|---|---|
| No. | 2θ (°) | Strength (%) |
| 1 | 7.2 | 31.5 |
| 2 | 8.0 | 9.5 |
| 3 | 8.4 | 14.1 |
| 4 | 9.1 | 7.5 |
| 5 | 11.7 | 12.4 |
| 6 | 12.4 | 12.7 |
| 7 | 14.1 | 26.0 |
| 8 | 16.8 | 40.3 |
| 9 | 18.1 | 100.0 |
| 10 | 20.6 | 40.5 |

### Example 22: Synthesis of Mesylate Crystal Form A of the Compound Represented by Formula I

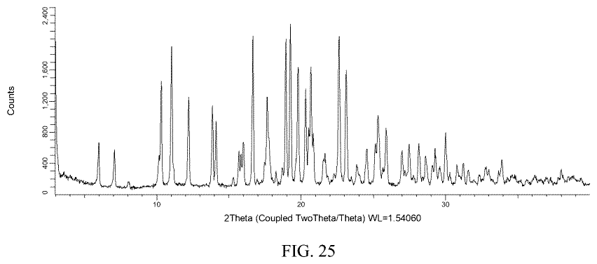

Weigh about 800 mg of the crystal form α of the compound represented by formula I and 104.73 mg of methanesulfonic acid, add 10 mL of acetonitrile solvent, stir magnetically at room temperature overnight, centrifuge to obtain a solid, and dry it under vacuum at 50° C. for 4 hours. The mesylate salt of the compound represented by formula I is obtained. It was identified through X-ray powder diffraction, showing that it is the mesylate crystal form A of the compound represented by formula I. Its XRPD spectrum is shown in FIG. 24, and the XRPD representative characteristic diffraction peak data is shown in Table 19.

TABLE 19
XRPD diffraction peaks of mesylate crystal form A
of the compound represented by formula I

| | diffraction angle | Relative |
|---|---|---|
| No. | 2θ (°) | Strength (%) |
| 1 | 7.3 | 52.2 |
| 2 | 10.5 | 100.0 |
| 3 | 15.1 | 44.5 |
| 4 | 15.5 | 49.2 |
| 5 | 20.9 | 41.8 |
| 6 | 21.4 | 80.4 |
| 7 | 21.9 | 38.9 |
| 8 | 22.2 | 60.7 |

### Example 23: Synthesis of Phosphate Crystal Form D of the Compound Represented by Formula I

Weigh about 800 mg of the crystal form α of the compound represented by formula I and 107.18 mg of phosphoric acid, add 10 mL of methanol solvent, stir magnetically at room temperature overnight, centrifuge to obtain a solid, and vacuum dry at 50° C. for 4 hours to obtain I compound phosphate. It was identified by X-ray powder diffraction, showing that it is the phosphate crystal form D of the compound represented by formula I. Its XRPD spectrum is shown in FIG. 25, and the XRPD representative characteristic diffraction peak data is shown in Table 20.

TABLE 20
XRPD diffraction peaks of phosphate crystal form D
of the compound represented by formula I

| | diffraction angle | Relative |
|---|---|---|
| No. | 2θ (°) | Strength (%) |
| 1 | 5.9 | 23.1 |
| 2 | 7.0 | 20.9 |
| 3 | 10.3 | 61.6 |
| 4 | 11.0 | 82.0 |
| 5 | 12.2 | 51.7 |
| 6 | 13.8 | 49.0 |
| 7 | 14.1 | 39.7 |
| 8 | 16.6 | 94.4 |
| 9 | 17.6 | 51.6 |
| 10 | 18.9 | 90.5 |
| 11 | 19.2 | 100.0 |
| 12 | 19.7 | 74.6 |
| 13 | 20.3 | 61.5 |
| 14 | 20.6 | 74.5 |
| 15 | 22.6 | 94.8 |
| 16 | 23.1 | 68.3 |

### Example 24: Synthesis of Hydrochloride Crystal Form A of the Compound Represented by Formula I

Weigh about 60 g of the crystal form α of the compound represented by Formula I and dissolve it in 150 mL of dichloromethane and 20 mL of methanol solvent. After spinning to dryness, add 200 mL of dichloromethane to dissolve, and slowly add 22.49 mL of 4M hydrochloric acid dioxane solution dropwise in an ice bath. After the dropwise addition is completed, spin off about 40 mL of methylene chloride, sonicate for 10 minutes, and filter to separate. The filter cake was rinsed with 100 mL of methylene chloride and dried under vacuum to constant weight to obtain 54 g of the hydrochloride of the compound represented by Formula I. It was identified by X-ray powder diffraction, showing that it is the hydrochloride crystal form A of the compound represented by formula I. Its XRPD spectrum is shown in FIG. 15, and the XRPD representative characteristic diffraction peak data is shown in Table 21.

TABLE 21
XRPD diffraction peaks of hydrochloride crystal form A
of the compound represented by formula I

| | diffraction angle | Relative |
|---|---|---|
| No. | 2θ (°) | Strength (%) |
| 1 | 6.0 | 35.2 |
| 2 | 7.4 | 17.5 |
| 3 | 11.0 | 22.2 |
| 4 | 13.8 | 48.7 |
| 5 | 14.2 | 97.4 |
| 6 | 16.1 | 55.1 |
| 7 | 18.1 | 100.0 |
| 8 | 18.5 | 98.9 |
| 9 | 20.1 | 64.2 |
| 10 | 21.4 | 72.3 |
| 11 | 23.1 | 23.7 |
| 12 | 23.9 | 60.0 |
| 13 | 24.0 | 46.2 |
| 14 | 25.6 | 73.7 |

### Example A: L-Malic Acid Content Detection

Inspection method: Ion chromatography (“Chinese Pharmacopoeia” 2020 edition, Part 4, General Chapter 0513)
Instrument: Thermo ICS-2100 ion chromatograph
Column: Dionex Ionpac® AS11-HC Analytical Column, 4.0×250 mm
Guard column: Dionex Ionpac® AG11-HC, 4×50 mm
Suppressor: ASRS 300-4 mm or AERS 500-4 mm
Flow rate: 1.0 mL/min
Injection volume: 10 μL
Column temperature: 30° C.
Injection mode: Pushseq Full
Eluent: 15 mmol/L potassium hydroxide solution
Current: 38 mA
Thinner: water
Running time: approximately 1.2 times the retention time of the principal component

### Specific Experimental Operations:

Preparation of the test solution: Take about 20 mg of the crystal form A of the compound shown in formula III (Example 4, Method 1), weigh it accurately, put it in a 100 mL measuring bottle, add an appropriate amount of water, dissolve it with ultrasound, and dilute it with water to Scale, shake well, and use as test solution.

Preparation of the reference substance solution: Take about 30 mg of the L-malic acid reference substance, weigh it accurately, place it in a 100 mL measuring bottle, add an appropriate amount of water, sonicate to dissolve, dilute with water to the mark, shake well, and accurately measure 1 mL Place in a 10 mL volumetric flask, dilute with water to the mark, shake well, and use it as a reference solution.

Determination method: Precisely measure 10 μL each of the test solution and the reference solution, inject them into the ion chromatograph respectively, record the chromatogram, and calculate the peak area according to the external standard method.

### Calculation Formula

$$f = \frac{Weighing volume of the control \times Control Content}{Peak area of control \times Dilution of control}$$

$$L - Malic acid content ( \% ) = \frac{\begin{matrix}Peak area of L - malic acid in the test product \times \\ {f}_{average} \times Dilutions of the test product\end{matrix}}{\begin{matrix}Weighing volume of test product \times \\ ( 1 - Moisture of the test product \% )\end{matrix}} \times 100 \%$$

The test calculation results are shown in Table 22:

TABLE 22

| | Actual measured | |
|---|---|---|
| | L-malic acid | Average L-malic |
| sample | content (%) | acid content (%) |
| Crystal form A of | 15.58 | 15.41 |
| the compound | 15.52 | |
| represented by | 15.64 | |
| formula III | 14.89 | |

According to the results obtained by the ion chromatograph in the above table, it can be concluded that the molar ratio between the free base (compound shown in Formula I) and L-malic acid in the crystal form A of the compound shown in Formula III is approximately 1:1.

### Example B: Single Crystal Test

Measured according to the first method of Part Four General Chapter 0451 of the 2020 edition of the “Chinese Pharmacopoeia”, test conditions: MoKα, λ=0.71073 Å.

Single crystal diffractometer: Bruker D8 Venture single crystal diffractometer. Single crystal diffraction data were collected from the sample prepared by Method 3 of Example 4. The single crystal structure analysis results showed that the obtained single crystal was a monohydrate, and the corresponding theoretical moisture content was 2.03%. The single crystal structure information is summarized in Table 23. The ellipsoid diagram of its molecular structure is shown in FIG. 5-3.

TABLE 23
Summary table of single crystal structure information of compound
form A represented by formula III

| | |
|---|---|
| Molecular formula | C₄₀H₅₄BrN₈O₈P |
| molecular weight | 885.79 |
| Crystal system | triclinic system |
| space group | P1 |
| Cell parameters | a = 11.4150(9) Å, b = 11.7403(10) Å, |
| | c = 16.6511(13) Å, |
| | α = 104.889(3)°, β = 94.070(3)°, γ = 91.714(3)° |
| unit cell volume | V = 2148.5(3) Å³ |
| Calculate density | 1.369 g/cm³ |

### Example C: Moisture Content Determination

Use the Mettler-Toledo coulometric Karl Fischer titrator to measure C20. Add an appropriate amount of cathode and anode sharing solution to the test cell and the diaphragm-free electrolytic electrode respectively. After the moisture titrator reaches equilibrium, weigh about 100 (±10) mg test sample into the test pool, record the measured moisture content, and conduct three parallel tests. Read the moisture content in the test product directly from the display data of the instrument, and calculate the average of the three moisture content results of the test product.

The measured value of moisture content can be found in the Examples of Crystal Form Preparation. For the crystal form A of the compound shown in formula III, the theoretical contents corresponding to different molar equivalents of moisture are as shown in Table 24:

TABLE 24

| molar equivalent | Content percentage |
|---|---|
| 0.5 mol | 1.03% |
| 1.0 mol | 2.03% |
| 1.5 mol | 3.02% |
| 2.0 mol | 3.98% |
| 2.5 mol | 4.93% |

### Example D: DSC Test

The instruments and parameters of the DSC test are shown in Table 25:

TABLE 25

| Device name | Differential scanning calorimetry | |
|---|---|---|
| | (DSC) | |
| Device model | Discovery DSC 2500 | |
| Sample tray | Aluminum crucible | |
| Protective gas | Nitrogen | |
| Gas flow rate | 50 | mL/min |
| Heating rate | 10° | C./min |

| | |
|---|---|
| temperature | 30° C.-Set end point temperature |
| range | |

### Example E: DVS Test

The instruments and parameters of the DVS test are shown in Table 26:

TABLE 26

| Device name | Dynamic Vapor Sorption (DVS) | |
|---|---|---|
| Manufacturer | Surface Measurement Systems | |
| Device model | DVS Resolution | |
| Sample tray | Aluminum crucible | |
| Protective gas | Nitrogen | |
| Gas flow rate | 200 | sccm |
| Detect temperature | 25° | C. |
| dm/dt | 0.002%/min | |
| Minimum dm/dt | 5 | min |
| balancing time | | |
| Maximum balancing | 360 | min |

| | |
|---|---|
| time | |
| RH gradient | 10% (50% RH-95% RH, 95% |
| | RH-0% RH-95% RH) |

Take crystal form A of the compound represented by Formula III for DVS measurement, and the obtained DVS spectrum is shown in FIG. 5-2. The DVS results show that the hygroscopic weight gain of the sample at 25° C./80% RH is about 3.43%, and the crystal form of the sample does not change before and after the test; in addition, the sample is placed in DVS at 25° C./80% RH for 24 hours, and the result shows the sample weight It remained basically unchanged and the crystal form did not change after 24 hours.

### Control Example 1

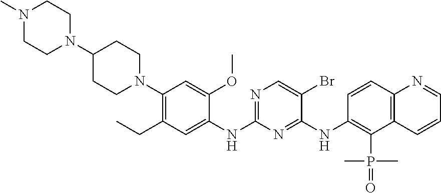 [SMILES: CCc1cc(Nc2ncc(Br)c(Nc3ccc4ncccc4c3P(C)(C)=O)n2)c(OC)cc1N1CCC(N2CCN(C)CC2)CC1]

The above compounds were prepared according to compound 41 of WO2019015655A1.

### Test 1: Kinase Inhibition Test

Since all the compounds of the present invention, including the crystal form of the compound represented by formula I and the salt and crystal form of the compound represented by formula I, have the same active ingredients as the free base, their kinase inhibitory activity is similar to that of the free base, among which PCT International application PCT/CN2021/075994 has recorded the kinase inhibitory activity of the free base, specifically:

Mobility shift assays were performed to determine the compound's inhibitory activity against EGFRΔ19del/T790M/C797S, EGFR WT, and IGF1R kinases. The enzyme reaction scheme is as follows:
1. Prepare 1*kinase buffer as follows.

| | Final |
|---|---|
| 1* Kinase buffer | concentration |
| HEPES PH 7.5 (mM) | 50 |
| Brij-35 | 0.0150% |
| DTT (mM) | 2 |
| Mgcl₂, Mncl₂ (mM) | 10 |

2. Preparation of compound concentration gradient: The starting concentration of the test compound is 3000 nM or 100 nM, dilute it in the 384 source plate to a 100% DMSO solution of 100 times the final concentration, and use precision to dilute the compound 3 times to 10 concentrations. Use the Dispenser Echo 550 to transfer 250 nL of 100× the final concentration of compound to the destination plate OptiPlate-384F.
3. Prepare a kinase solution with 2.5 times the final concentration using 1× Kinase buffer.
4. Add 10 μL of 2.5 times the final concentration of kinase solution to the compound wells and positive control wells respectively; add 10 μL of 1× Kinase buffer to the negative control wells.
5. Centrifuge at 1000 rpm for 30 seconds, shake and mix the reaction plate and incubate at room temperature for 10 minutes.
6. Use 1× Kinase buffer to prepare a mixed solution of ATP and Kinase substrate at 5/3 times the final concentration.
7. Add 15 μL of a mixed solution of ATP and substrate at 5/3 times the final concentration to start the reaction.
8. Centrifuge the 384-well plate at 1000 rpm for 30 seconds, mix well by shaking, and incubate at room temperature for the corresponding time.
9. Add 30 μL of stop detection solution to stop the kinase reaction, centrifuge at 1000 rpm for 30 seconds, and shake to mix.
10. Use Caliper EZ Reader to read conversion rates.
11. Calculation formula

$$\% Inhibitions = \frac{Conversion \% \_max - Conversion \% \_sample}{Conversion \% \_max - Conversion \% \_min} \times 100$$

Among them: Conversion %_sample is the conversion rate reading of the sample; Conversion %_min: the mean value of the negative control wells, representing the conversion rate reading of the wells without enzyme activity; Conversion %_max: the mean value of the positive control wells, representing the conversion rate reading of the wells without compound inhibition.

The fitted dose-response curve uses the log value of concentration as the IC50 value of enzyme activity.

The calculation formula is Y=Bottom+(Top−Bottom)/(1+10\hat{ }((Log IC50−X)*HillSlope)).

The results are expressed as IC₅₀ values, as shown in Table 27.

TABLE 27

| | EGFR | | |
|---|---|---|---|
| | Δ19del/T790M/C797S | EGFR WT | IGF1R |
| compound | IC₅₀ (nM) | IC₅₀ (nM) | IC₅₀ (nM) |
| Control Example 1 | 0.2 | 5.1 | 9.5 |
| Amorphous material | 0.3 | 20.2 | 195 |
| represented by | | | |
| formula I | | | |

Test 2: Cell Proliferation Test Since all the compounds of the present invention, including the crystal form of the compound represented by formula I and the salt and crystal form of the compound represented by formula I, possess the same active ingredients as the free base, their cell proliferation inhibitory activity is similar to that of the free base, among which PCT International application PCT/CN2021/075994 has recorded the cell proliferation inhibitory activity of free bases, specifically:

### 1. Cell Culture

Cell Lines: Suspension cells: Ba/F3 cells with stable overexpression of the Δ 19del/T790M/C797S mutant gene, named Ba/F3-Δ 19del/T790M/C797S; cells overexpressing EGFR WT, named Ba/F3 EGFR WT;
Adherent cells: human epidermal cancer cell A431 carrying EGFR WT

### A. Culture Medium

RPMI 1640 with 10% FBS and 1% penicillin or DMEM with 10% FBS and 1% penicillin

### B. Cell Recovery

a) Preheat the medium in a 37° C. water bath.
b) Take out the cryovial from the liquid nitrogen tank, quickly put it into a 37° C. water bath, and allow it to completely melt within 1 minute.
c) Transfer the cell suspension to a 15 mL centrifuge tube containing 8 mL of culture medium and centrifuge at 1000 rpm for 5 min.
d) Discard the supernatant, resuspend the cells in 1 mL of culture medium, transfer to a 75 cm2 culture flask containing 15 mL of culture medium, add an appropriate volume of culture medium, and incubate in a 37° C., 5% CO₂ incubator culture in.

### C. Cell Passage

a) Preheat the medium in a 37° C. water bath.
b) Suspension cells Collect cells directly into a 15 mL centrifuge tube. After washing the adherent cells with PBS, add appropriate trypsin for digestion, add culture medium and pipet, transfer to a 15 mL centrifuge tube, and then centrifuge at 1000 rpm for 5 minutes. Discard the supernatant, resuspend the cells and pass them at the appropriate ratio, and place them in a 37° C., 5% CO₂ incubator.

### 2. Compound Preparation

a) Test compounds (20 mM stock solution) were diluted with 100% DMSO as a starting concentration to 10 mM, and then the compounds were diluted 3-fold into 12 concentration gradients each (Cat #P-05525, Labcyte);
b) Dilute the above compound solution 100 times with culture medium to prepare a 10-fold working solution;

### 3. Cell Seeding in 96-Well Plate

a) Centrifuge cells growing in the logarithmic phase at 1000 rpm for 5 minutes, discard the supernatant, resuspend the cells in culture medium, and then count the cells;
b) Seed cells into a 96-well cell culture plate at a density of 2000 or 3000 cells/well, 135 L/well.

### 4. Compound Handling

a) Add 15 μL of the compound prepared in step 2 into the cell plate per well. The final maximum concentration is 10000 nM or 1111 nM, 9 concentration gradients, 3-fold dilution, and the final concentration of DMSO is 0.1%. The blank control well is culture medium (0.1% DMSO);
b) Incubate the cells in the incubator for another 72 hours.

### 5. Testing

a) Take out the 96-well cell culture plate and add 50 μL CTG reagent (CellTiter Glo kit, promega, Cat #G7573).
b) Shake for 2 minutes and react at room temperature for 10 minutes.
c) Use PerkinElmer reader to read the luminescence signal value Lum.

### 6. Experimental Data Processing

Calculate the cell survival inhibition rate of each well, use GraphPad Prism 6.0 software to analyze the data, use a nonlinear regression equation to fit the data to obtain a dose-effect curve, and calculate the compound IC₅₀:

$$Cell survival inhibition rate ( \% ) = ( 1 - ( {Lum}_{test compound} - {Lum}_{medium control} ) / ( {Lum}_{cell control} - Lum culture medium control ) ) \times 100 \%$$

$$Y = minimum value + ( maximum value - minimum value ) / ( 1 + {10}^{⋀} ( ( {LogIC}_{50} - X ) * slope ) ) ;$$

X: logarithm of compound concentration; Y: cell survival inhibition rate.

Cell proliferation assay results are expressed as IC₅₀, as shown in Table 28.

TABLE 28

| | Ba/F3/EGFR | BaF3 EGFR | |
|---|---|---|---|
| | Δ19del/T790M/C797S | WT | A431 |
| compound | IC₅₀ (nM) | IC₅₀ (nM) | IC₅₀ (nM) |
| Control Example 1 | 9 | 39.9 | 245 |
| Amorphous material | 6.8 | 151 | 1128 |
| represented by | | | |
| formula I | | | |

### Test 3: Measurement of Crystal Form Stability

The X-ray powder diffraction pattern detection equipment and method of the present invention are as shown in the X-ray powder diffraction table in the instrument and analysis method. After the test compounds are placed under different temperature, humidity and light conditions for a period of time, the purity is tested. Purity testing method: Use high-performance liquid chromatography (HPLC) to detect the chemical purity of this product. Determine according to high performance liquid chromatography (“Chinese Pharmacopoeia” 2020 edition, Part IV, General Chapter 0512). Use octadecylsilane bonded silica gel as the filler (Waters XBridge Shield RP18 (4.6×250 mm, 5 μm)), 0.1% triethanolamine, 0.01 mol/L potassium dihydrogen phosphate solution (use phosphoric acid to adjust the pH value to 2.8) is mobile phase A, and acetonitrile is mobile phase B. The detection wavelength is 220 nm, the flow rate is 1.0 mL/min, and the column temperature is 35° C. The detection results are detailed in Table 29. XRPD characterization was performed on the samples after being placed under different temperature, humidity and light conditions for a period of time. The XRPD detection spectrum (in addition to the compounds in Table 28, the test substances also include the crystal form of the compound shown in Formula I) is basically the same.

TABLE 29
Chemical stability test results of different crystal forms

| | | | | | Chemical |
|---|---|---|---|---|---|
| | | Chemical | Chemical | Chemical | purity (%) |
| | Placement | purity (%) | purity (%) | purity (%) | 4500 lx |
| Compound crystal form | time | 0 days | 60° C. | 92.5% RH | illumination |
| Hydrochloride crystal form B | 9 days | 99.26 | 98.63 | 99.21 | 98.71 |
| (Example 18) | | | | | |
| Succinate crystal form A | 15 days | 99.25 | 98.47 | 99.20 | 98.31 |
| (Example 21) | | | | | |
| Phosphate crystal form D | 9 days | 99.36 | 98.96 | 99.37 | 98.72 |
| (Example 23) | | | | | |
| L-tartrate crystal form A | 9 days | 98.86 | 98.52 | 98.94 | 98.16 |
| (Example 19) | | | | | |
| Mesylate crystal form A | 9 days | 99.07 | 98.66 | 98.91 | 98.30 |
| (Example 22) | | | | | |
| Fumarate crystal form B | 9 days | 98.67 | 98.16 | 98.56 | 97.15 |
| (Example 20) | | | | | |
| Crystal form A of the | 13 days | 98.85 | 98.80 | 98.98 | 98.83 |
| compound represented by | | | | | |
| formula III | | | | | |
| (Example 4, Method 1) | | | | | |
| Crystal form J of the | 10 days | 99.59 | 98.61 | 99.59 | 96.05 |
| compound represented by | | | | | |
| formula III | | | | | |

### Test 4: Pharmacokinetic Test

Male SD rats purchased from Beijing Weitonglihua Experimental Animal Technology Co Ltd. were used for oral administration PK studies. The dose was 50 mg/kg, the preparation was distilled water, and the compound concentrations corresponding to the test compounds were 5 mg/mL, ready for use. Oral administration is by oral gavage at 10 mL/kg. Blood was collected through the rat orbital venous plexus, and 300 L was taken at each time point into an EDTA anticoagulant tube. The blood collection time is: 15 min, 30 min, 1 h, 2 h, 4 h, 7 h, 24 h, 30 h, 48 h, centrifuge at 4000 rpm for 10 min, take the supernatant to obtain 100 μL plasma, and store it in a −80° C. refrigerator, spare. After precipitating the protein from the above plasma sample with acetonitrile, take the supernatant and mix it with water 1:1, and take 10 μL for LC-MS/MS detection. The results are shown in Table 30.

TABLE 30
Pharmacokinetic test results

| | Dosing | dosage | Cₘₐₓ | AUCₗₐₛₜ |
|---|---|---|---|---|
| compound | method | (mg/kg) | (ng/ml) | (h*ng/ml) |
| Crystal form A of the | PO | 50 | 2557 | 47593 |
| compound represented by | | | | |
| formula III | | | | |
| (Example 4, Method 1) | | | | |
| Crystal form J of the | PO | 50 | 1993 | 27401 |
| compound represented by | | | | |
| formula III | | | | |
| (Example 13) | | | | |
| Hydrochloride crystal | PO | 50 | 2420 | 39522 |
| form B | | | | |
| (Example 18) | | | | |
| L-tartrate crystal form A | PO | 50 | 2657 | 45429 |
| (Example 19) | | | | |
| Fumarate crystal form B | PO | 50 | 2803 | 45794 |
| (Example 20) | | | | |
| Succinate crystal form A | PO | 50 | 2733 | 45707 |
| (Example 21) | | | | |
| Phosphate crystal form D | PO | 50 | 2677 | 43648 |
| (Example 23) | | | | |

1. A crystal form of a compound shown in Formula I, characterized in that the crystal form is selected from any one or more of crystal form α, crystal form β, crystal form γ and crystal form δ; 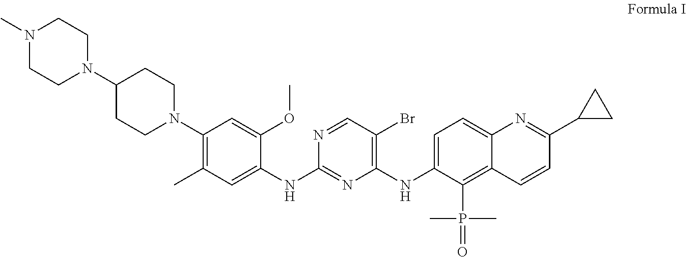
wherein, the X-ray powder diffraction pattern of Form α is substantially as shown in FIG. 1;
the crystalline form β is characterized by having an X-ray powder diffraction pattern comprising characteristic peak in term of angle 2θ at 4.7±0.2°, 10.3±0.2°, 11.2±0.2°, 11.6±0.2°, 13.1±0.2°, 13.3±0.2°, 14.5±0.2°, 17.5±0.2°, 18.6±0.2°, 18.9±0.2°, 19.7±0.2°, 20.3±0.2°, 21.4±0.2°, 21.8±0.2°; or, all the X-ray powder diffraction pattern of the crystal form β is basically shown in FIG. 2;
the crystalline form γ is characterized by having an X-ray powder diffraction pattern comprising characteristic peak in term of angle 2θ at 4.8±0.2°, 7.6±0.2°, 9.8±0.2°, 10.0±0.2°, 11.6±0.2°, 19.8±0.2°, 4.8±0.2°, 7.6±0.2°, 9.8±0.2°, 10.0±0.2°, 11.6±0.2°, 14.3±0.2°, 14.8±0.2°, 15.5±0.2°, 19.1±0.2°, 19.5±0.2°, 19.8±0.2°, 20.0±0.2°, 22.2±0.2°, 23.1±0.2°, 23.9±0.2°; or, the X-ray powder diffraction pattern of the crystal form γ is basically shown in FIG. 3;
the crystalline form δ is characterized by having an X-ray powder diffraction pattern comprising characteristic peak in term of angle 2θ at 5.9±0.2°, 8.2±0.2, 9.6±0.2°, 10.7±0.2°, 11.2±0.2°, 15.7±0.2°, 21.8±0.2°; or, the X-ray powder diffraction pattern of the crystal form δ is basically shown in FIG. 4.

2. A salt of a compound represented by Formula I, wherein the structure of Formula I is:

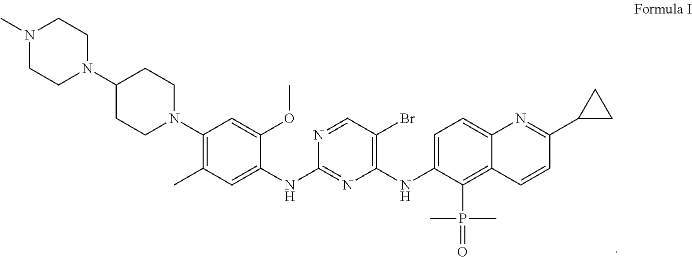 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1]

3. The salt of the compound of Formula I according to claim 2, characterized in that the salt is malate, hydrochloride, phosphate, tartrate, fumarate, succinate or methanesulfonic acid salt.

4. The salt of the compound of Formula I according to claim 3, characterized in that the malate is L-malate.

5. The salt of the compound shown in Formula I according to claim 4, characterized in that it has the structure of the compound shown in Formula II: 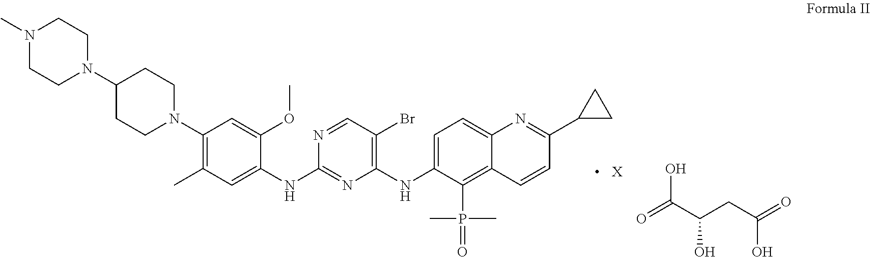
wherein, x is selected from 0.5-5; or, x is selected from 0.5-3.0, or 0.8-3.0; or, 1.0, 2.0 or 3.0; or x is selected from 0.5, 0.8, 1.0, 1.2, 1.5, 1.8, 2.0, 2.2, 2.5, 2.8, 3.0, 3.2, 3.5, 3.8, 4.0, 4.2, 4.5, 4.8, 5.0 or any other value within the range of 0.5-5.

6. (canceled)

7. The salt of the compound shown in Formula I according to claim 5, characterized in that the compound shown in Formula II is a compound shown in Formula III: 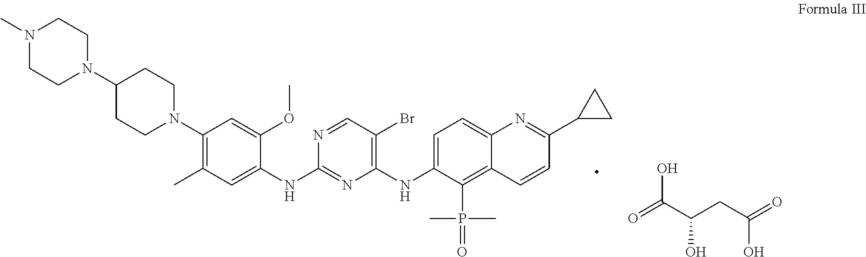 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1 ; O=C(O)C[C@H](O)C(=O)O]
wherein the compound represented by Formula III is amorphous or crystalline, wherein the crystalline form of said compound shown in Formula III is selected from any one or more of crystalline form A, crystalline form B, crystalline form C, crystalline form D, crystalline form E, crystalline form F, crystalline form G, crystalline form H, crystalline form I, and crystalline form J.

8. (canceled)

9. (canceled)

10. The salt of the compound represented by Formula I according to claim 7, characterized in that the crystalline form A is characterized by having an X-ray powder diffraction pattern comprising characteristic peak in term of angle 2θ at 5.5±0.2°, 8.3±0.2°, 15.1±0.2° and 17.9±0.2°; or, the X-ray powder diffraction pattern of crystalline form A comprises one or more of the following diffraction angles 2θ: 7.8±0.2°, 9.2±0.2°, 11.3±0.2°, 11.7±0.2°, 13.6±0.2°, 13.8±0.2°, 16.4±0.2°, 16.6±0.2°, 17.2±0.2°, 20.1±0.2°, 20.9±0.2°; or, comprising characteristic peaks of 5.5±0.2°, 8.3±0.2°, 13.8±0.2°, 15.1±0.2°, 16.6±0.2° and 17.9±0.2°; or, comprising characteristic peaks of 5.5±0.2°, 8.3±0.2°, 13.6±0.2°, 13.8±0.2°, 15.1±0.2°, 16.6±0.2° and 17.9±0.2°; or, comprising characteristic peaks of 5.5±0.2°, 7.8±0.2°, 8.3±0.2°, 9.2±0.2°, 11.3±0.2°, 11.7±0.2°, 13.6±0.2°, 13.8±0.2°, 15.1±0.2°, 16.4±0.2°, 16.6±0.2°, 17.2±0.2°, 17.9±0.2°, 20.1±0.2°, 20.9±0.2°; or, the X-ray powder diffraction pattern of the crystal form A is basically shown in FIG. 5.

11. The salt of the compound of Formula I according to claim 10, characterized in that the crystal form A is a hydrate.

12. The salt of the compound of Formula I according to claim 11, characterized in that the crystal form A contains y molar equivalents of water, and the y is selected from 0.5 to 4.0; or the y is selected from 0.5 to 2.5; or, the said y is selected from 1.0-2.5; or, y is 1.0.

13. (canceled)

14. The salt of the compound represented by Formula I according to claim 11, characterized in that the moisture content contained in the crystal form A of the compound represented by Formula III is 1%-5%; or, the moisture content contained in the crystalline form A of the compound shown in the Formula III is 1%-4%; or, the moisture content contained in the crystalline form A of the compound shown in Formula III is 1.0%-3.70%; or, the moisture content contained in the crystalline form A of the compound shown in Formula III is 2.0%-3.7%.

15. The salt of the compound represented by Formula I according to claim 7, the crystalline form B is characterized by having an X-ray powder diffraction pattern comprising characteristic peak in term of angle 2θ at 5.6±0.2°, 10.0±0.2°, 11.1±0.2°, 13.0±0.2°, 13.7±0.2°, 14.4±0.2°, 18.0±0.2°, 19.0±0.2°, 20.2±0.2°, and 20.6±0.2°; or, the X-ray powder diffraction spectrum of the crystal form B is basically as shown in FIG. 6, or
the crystalline form C is characterized by having an X-ray powder diffraction pattern comprising characteristic peak in term of angle 2θ at 7.2±0.2°, 8.4±0.2°, 9.2±0.2°, 11.6±0.2°, 12.3±0.2°, 14.2±0.2°, 16.8±0.2°, 18.0±0.2°, and 20.6±0.2°; or, the X-ray powder diffraction pattern of the crystal form C is basically shown in FIG. 7; or
the crystalline form D is characterized by having an X-ray powder diffraction pattern comprising characteristic peak in term of angle 2θ at 5.4±0.2°, 8.3±0.2°, 14.8±0.2°, 16.4±0.2°, and 17.6±0.2°; or, the X-ray powder diffraction pattern of the crystal form D is basically as shown in FIG. 8; or
the crystalline form E is characterized by having an X-ray powder diffraction pattern comprising characteristic peak in term of angle 2θ at 7.1±0.2°, 11.9±0.2°, 14.3±0.2°, 15.1±0.2°, 15.9±0.2°, 19.3±0.2° and 20.5±0.2°; or, the X-ray powder diffraction pattern of the crystal form E is basically shown in FIG. 9; or
the crystalline form F is characterized by having an X-ray powder diffraction pattern comprising characteristic peak in term of angle 2θ at 6.6±0.2°, 7.4±0.2°, 10.5±0.2°, 16.4±0.2°, and 21.1±0.2°; or, the X-ray powder diffraction pattern of the crystal form F is basically as shown in FIG. 10; or
the crystalline form G is characterized by having an X-ray powder diffraction pattern comprising characteristic peak in term of angle 2θ at 5.0±0.2°, 10.0±0.2°, 15.0±0.2°, and 19.5±0.2°; or, the X-ray powder diffraction pattern of the crystalline form G is basically shown in FIG. 11; or
the crystalline form H is characterized by having an X-ray powder diffraction pattern comprising characteristic peak in term of angle 2θ at 4.7±0.2°, 9.3±0.2°, and 14.0±0.2°; or,
the X-ray powder diffraction pattern of the crystalline form H is basically shown in FIG. 12; or
the X-ray powder diffraction pattern of Form I is substantially as shown in FIG. 13; or
the crystalline form J is characterized by having an X-ray powder diffraction pattern comprising characteristic peak in term of angle 2θ at 9.0±0.2°, 11.2±0.2°, 11.7±0.2°, 12.2±0.2°, 14.0±0.2°, 15.5±0.2°, 16.2±0.2°, 18.0±0.2°, 19.2±0.2°, and 20.0±0.2°; or, the X-ray powder diffraction spectrum of the crystal form J is essentially shown in FIG. 14.

16. (canceled)

17. (canceled)

18. (canceled)

19. (canceled)

20. (canceled)

21. (canceled)

22. (canceled)

23. (canceled)

24. The salt of the compound represented by Formula I according to claim 5, characterized in that x is selected from 2.0, and its structure is shown in Formula IV: 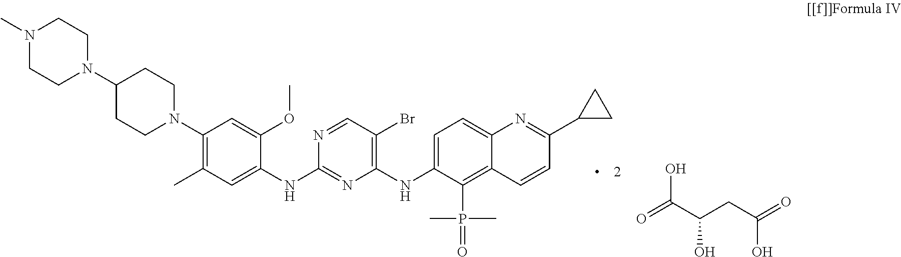 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1 ; "2" ; O=C(O)C[C@H](O)C(=O)O]
wherein the compound represented by Formula IV is amorphous or crystalline, wherein the crystal form of the compound represented by Formula IV is any one or more of crystal form A, crystal form B, and crystal form C.

25. (canceled)

26. (canceled)

27. The salt of the compound represented by Formula I according to claim 24, the crystalline form A is characterized by having an X-ray powder diffraction pattern comprising characteristic peak in term of angle 2θ at 5.5±0.2°, 6.2±0.2°, 6.5±0.2°, 9.1±0.2°, 9.4±0.2°, 11.2 0.2°, 13.1±0.2°, 13.4±0.2°, 15.1±0.2°, 18.0±0.2°, 18.2±0.2°, 19.5±0.2°, 20.4±0.2°, 21.2±0.2°, 21.3±0.2°, 21.7±0.2°, 23.3±0.2°, 24.9±0.2°; or, the X-ray powder diffraction spectrum of crystal form A is essentially shown in FIG. 16; or
the crystalline form B is characterized by having an X-ray powder diffraction pattern comprising characteristic peak in term of angle 2θ at 7.6±0.2°, 9.8±0.2°, 11.6±0.2°, 19.1±0.2°, 19.5±0.2°, 19.8±0.2°, 21.3±0.2°, 22.2±0.2°, 23.1±0.2°; or, the X-ray powder diffraction spectrum of crystal form B is essentially shown in FIG. 17; or
the crystalline form C is characterized by having an X-ray powder diffraction pattern comprising characteristic peak in term of angle 2θ at 8.0±0.2°, 8.7±0.2°, 12.3±0.2°, and 21.9±0.2°; or, the X-ray powder diffraction pattern of Form C is essentially shown in FIG. 18.

28. (canceled)

29. (canceled)

30. The salt of the compound shown in Formula I according to claim 5, characterized in that, x is selected from 3.0, and its structure is shown in Formula V: 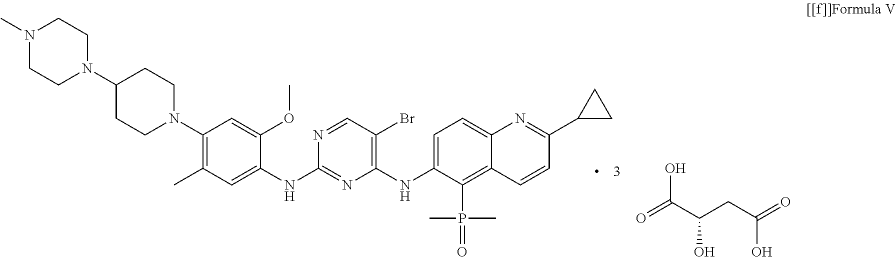 [SMILES: COc1cc(N2CCC(N3CCN(C)CC3)CC2)c(C)cc1Nc1ncc(Br)c(Nc2ccc3nc(C4CC4)ccc3c2P(C)(C)=O)n1 ; "3" ; O=C(O)C[C@H](O)C(=O)O]
wherein the compound represented by Formula V is amorphous or crystalline, wherein the crystal form of the compound represented by Formula V is crystal form A,
wherein the crystalline form A is characterized by having an X-ray powder diffraction pattern comprising characteristic peak in term of angle 2θ at 6.4±0.2°, 7.4±0.2°, 9.7±0.2°, 11.4±0.2°, 12.7±0.2°, 16.7±0.2°, 18.0±0.2°, 19.0±0.2°, 20.5±0.2°, 21.0±0.2°, 22.2±0.2°, 23.0±0.2°; or,
the X-ray powder diffraction spectrum of the crystal form A is substantially as shown in FIG. 19.

31. (canceled)

32. (canceled)

33. (canceled)

34. The salt of the compound shown in Formula I according to claim 2, characterized in that the salt of the compound shown in Formula I is a hydrochloride, wherein the molar ratio of the compound shown in Formula I and hydrochloric acid is 1:1,
wherein the compound represented by Formula I is amorphous or crystalline, wherein the crystal form of the hydrochloride of the compound represented by Formula I is one of crystal form A, crystal form B or a mixture thereof, or
wherein the crystalline form A is characterized by having an X-ray powder diffraction pattern comprising characteristic peak in term of angle 2θ at 6.0±0.2°, 7.4±0.2°, 11.0±0.2°, 13.8±0.2°, 14.2±0.2°, 16.1±0.2°, 18.1±0.2°, 18.5±0.2°, 20.1±0.2°, 21.4±0.2°, 23.1±0.2°, 23.9±0.2°, 24.0±0.2°, 25.6±0.2°, or, the X-ray powder diffraction spectrum of the crystal form A is substantially as shown in FIG. 15; or
the crystalline form B is characterized by having an X-ray powder diffraction pattern comprising characteristic peak in term of angle 2θ at 6.6±0.2°, 7.1±0.2°, 9.2±0.2°, 11.4±0.2°, 12.5±0.2°, 13.1±of 0.2°, 19.3±0.2°, 23.7±0.2°, 24.0±0.2°, and 26.5±0.2°; or, the X-ray powder diffraction spectrum of the crystal form B is basically as shown in FIG. 20; or
characterized in that the salt of the compound shown in Formula I is tartrate, wherein the tartrate is L-tartrate, wherein the L-tartrate of the compound shown in Formula I is amorphous or crystalline, wherein the crystal form of the compound L-tartrate shown in Formula I is crystal form A; wherein, the crystalline form A of the L-tartrate is characterized by having an X-ray powder diffraction pattern comprising characteristic peak in term of angle 2θ at 5.8±0.2°, 7.0±0.2°, 9.9±0.2°, 11.7±0.2°, 12.6±0.2°, 14.0±0.2°, 17.8±0.2°, and 18.9±0.2°; or, the X-ray powder diffraction pattern of the crystal form A of the L-tartrate is basically shown in FIG. 21; or
characterized in that the salt of the compound shown in Formula I is fumarate, wherein the fumarate salt of the compound represented by Formula I is amorphous or crystalline, wherein the crystal form of the fumarate is crystal form B: wherein, the crystalline form B of the fumarate is characterized by having an X-ray powder diffraction pattern comprising characteristic peak in term of angle 2θ at 7.2±0.2°, 8.1±0.2°, 8.4±0.2°, 9.2±0.2°, 14.3±0.2°, 17.0±0.2°, 18.1±0.2°, and 20.7±0.2°; or, the X-ray powder diffraction pattern of the crystal form B of the fumarate is basically as shown in FIG. 22; or
characterized in that the salt of the compound shown in Formula I is succinate, wherein the succinate of the compound of Formula I is amorphous or crystalline, wherein the crystal form of the succinate is Form A; wherein, the crystalline form A of the succinate is characterized by having an X-ray powder diffraction pattern comprising characteristic peak in term of angle 2θ at 7.2±0.2°, 8.0±0.2°, 8.4±0.2°, 9.1±0.2°, 11.7±0.2°, 12.4±0.2°, 14.1±0.2°, 16.8±0.2°, 18.1±0.2°, and 20.6±0.2°; or, the X-ray powder diffraction spectrum of the crystal form A of the succinate is basically as shown in FIG. 23; or
characterized in that, the salt of the compound shown in Formula I is methanesulfonate, wherein the methanesulfonate salt of the compound represented by Formula I is amorphous or crystalline, wherein the crystal form of the mesylate is Form A; wherein the crystalline form A of the mesylate is characterized by having an X-ray powder diffraction pattern comprising characteristic peak in term of angle 2θ at 7.3±0.2°, 10.5±0.2°, 15.1±0.2°, 15.5±0.2°, 20.9±0.2°, 21.4±0.2° and 22.2±0.2°; or, the X-ray powder diffraction pattern of the crystal form A of the mesylate is basically shown in FIG. 24; or
characterized in that the salt of the compound of Formula I is a phosphate, wherein the phosphate salt of the compound represented by Formula I is amorphous or crystalline, wherein the crystal form of the phosphate is crystal form D, wherein the crystalline form D of the phosphate is characterized by having an X-ray powder diffraction pattern comprising characteristic peak in term of angle 2θ at 5.9±0.2°, 7.0±0.2°, 10.3±0.2°, 11.0±0.2°, 12.2±0.2°, 13.8±0.2°, 14.1±0.2°, 16.6±0.2°, 17.6±0.2°, 18.9±0.2°, 19.2±0.2°, 19.7±0.2°, 20.3±0.2°, 20.6±0.2°, 22.6±0.2°, 23.1±0.2°; or, the X-ray powder diffraction pattern of the crystal form D of the phosphate is basically shown in FIG. 25.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. A composition comprising therapeutically effective amount of the crystal form of the compound shown in Formula I according to claim 1 and pharmaceutically acceptable excipients.

60. A method for inhibiting various different forms of EGFR mutations, including one or more of L858R, Δ19del, T790M and C797S mutations, the method comprising administering the crystal form of the compound of Formula I according to claim 1 to a patient in need.

61. A method for treating EGFR-driven cancer, the method comprising administering to a patient in need a therapeutically effective amount of the crystal form of the compound represented by Formula I according to claim 1.

62. The method of claim 61, wherein the EGFR driven cancer is one or more mutations selected from the group consisting of: (i) C797S, (ii) L858R and C797S, (iii) C797S and T790M, (iv) L858R, T790M, and C797S, (v) Δ19del, T790M, and C797S, (vi) Δ19del and C797S, (vii) L858R and T790M, or (viii) Δ19del and T790M; or
the EGFR driven cancer is colon cancer, gastric cancer, thyroid cancer, lung cancer, leukemia, pancreatic cancer, melanoma, brain cancer, kidney cancer, prostate cancer, Ovarian or breast cancer, or
the lung cancer is non-small cell lung cancer carrying EGFR L858R/T790M/C797S or EGFR Δ19del/T790M/C797S mutation.

63. (canceled)

64. (canceled)

65. A method for inhibiting mutant EGFR in a patient, the method comprising administering to a patient in need a therapeutically effective amount of the crystal form of the compound represented by Formula I according to claim 1.

66. (canceled)

67. (canceled)

68. (canceled)

69. (canceled)
